(19) United States
(12) Patent Application Publication
(10) Pub. No.: US 2023/0384325 A1
(43) Pub. Date: Nov. 30, 2023

(54) NOVEL BIOMARKER OF ACUTE CORONARY SYNDROMES

(71) Applicant: UPSTREAM MEDICAL TECHNOLOGIES LIMITED, CHRISTCHURCH (NZ)

(72) Inventor: Christopher Joseph Pemberton, Christchurch (NZ)

(21) Appl. No.: 18/304,934

(22) Filed: Apr. 21, 2023

Related U.S. Application Data
(63) Continuation of application No. PCT/NZ2021/050185, filed on Oct. 22, 2021.
(60) Provisional application No. 63/104,917, filed on Oct. 23, 2020.

(51) Int. Cl. G01N 33/68; A61B 5/024; A61K 45/06

(52) U.S. Cl. CPC G01N 33/6893; A61B 5/02405; A61K 45/06; G01N 2333/4745; G01N 2800/324; G01N 2800/52

(57) ABSTRACT

The present invention is concerned with a novel circulating biomarker of cardiac disease. The present invention provides assays, methods and test kits for detection of insulin-like growth factor binding protein 3 and to the utility of IGFBP-3 in clinically relevant diagnoses of acute coronary syndromes including, for example, unstable angina pectoris, alongside classical risk factors including history of angina and abnormal electrocardiogram. The present invention further provides assays, methods and test kits for identifying inducible cardiac ischaemia in a patient by measuring the levels of IGFBP-3 in conjunction with other risk factors such as an elevated levels of troponin, an abnormal electrocardiogram, a history of heart failure and/or a history of myocardial alongside interrogation of the IGFBP-3 or ΔIGFBP-3 levels in a sample obtained from the patient, and using this information to inform prophylactic or therapeutic treatment options for ischaemia.

### CROSS REFERENCE TO RELATED APPLICATIONS

This application is a U.S. continuation patent application of International Patent Application No. PCT/NZ2021/050185, filed Oct. 22, 2021, which claims the benefit of U.S. Patent Application No. 63/104,917, filed Oct. 23, 2020, the entire contents of each of which are fully incorporated herein by reference.

### INCORPORATION BY REFERENCE OF MATERIAL SUBMITTED ELECTRONICALLY

A Sequence Listing, which is a part of the present disclosure, is submitted concurrently with the specification as a text file. The name of the text file containing the Sequence Listing is “56119A_Seqlisting.XML.” The Sequence Listing was created on Apr. 20, 2023, and is 3,021 bytes in size. The subject matter of the Sequence Listing is incorporated by reference herein in its entirety.

### TECHNICAL FIELD

The present invention is concerned with a novel circulating biomarker for cardiac disease. In particular, the present invention provides assays, methods and test kits for detection of insulin-like growth factor binding protein 3 (IGFBP-3) and to the utility of IGFBP-3 in combination with other clinical risk factors including, for example, history of angina and an abnormal electrocardiogram which may be used for identifying acute coronary syndromes in a patient including, for example, unstable angina pectoris.

### BACKGROUND OF THE INVENTION

The following includes information that may be useful in understanding the present invention. It is not an admission that any of the information, publications or documents specifically or implicitly referenced herein is prior art, or essential, to the presently described or claimed inventions. All publications and patents mentioned herein are hereby incorporated herein by reference in their entirety.

Approximately 65,000 patients present annually to hospital with chest pain in New Zealand making it one of the most common causes for presentation [1]. In these patients, the accurate and timely diagnosis of acute coronary syndromes (ACS, comprising myocardial infarction and unstable angina) is of major importance due to the high prevalence (˜25% of ACS patients), mortality and morbidity (6-fold increased risk of major adverse event within 2 years) associated with these conditions [2, 7]. The diagnosis of myocardial infarction is made on the basis of thorough clinical evaluation and the measurement of circulating cardiac troponins (cTn). More recently, the introduction of highly sensitive (hs) troponin assays has facilitated faster assessment pathways for myocardial infarction diagnosis [3-5]. In comparison, early diagnosis of unstable angina from clinical signs, symptoms, and blood tests is more difficult, often requiring time consuming invasive or provocative procedures such as angiography and stress testing [3]. As yet, no circulating biomarkers provide clinically useful information to aid the rapid diagnosis of unstable angina from other confounding diagnoses such as aortic dissection, pericarditis or pulmonary embolism and musculoskeletal chest pain [6].

Accordingly, there is a major unmet clinical need for markers that could rapidly distinguish unstable angina from other non-cardiac causes of chest pain, a problem that has major significance given that unstable angina is an important precursor for future myocardial infarction and has its own significant morbidity [7].

The present invention addresses this unmet clinical need through identification of a novel, circulating biomarker of acute coronary syndromes, including unstable angina.

### SUMMARY OF THE INVENTION

The inventions described and claimed herein have many attributes and embodiments including, but not limited to, those set forth or described or referenced in this Summary of the Invention. It is not intended to be all inclusive and the inventions described and claimed herein are not limited to or by the features or embodiments identified in this Summary of the Invention, which is included for purposes of illustration only and not restriction.

In an aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient at index presentation;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
wherein, an increase in the ΔIGFBP-3 in the patient sample relative to the reference standard from the control population is diagnostic that the patient has unstable angina pectoris.

In another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina in the patient, is diagnostic that the patient has unstable angina pectoris.

In another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina and a reduced heart rate relevant to a reference standard in the patient, is diagnostic that the patient has unstable angina pectoris.

In another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina and decreased levels of high-density lipoprotein relevant to a reference standard in the patient, is diagnostic that the patient has unstable angina pectoris.

In another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample measured relative to the reference standard from the control population, when measured together with a history of angina and an abnormal electrocardiogram relevant to a reference standard in the patient, is diagnostic that the patient has unstable angina pectoris.

In another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a diagnosis of ischaemia, optionally by imaging, in the patient, is diagnostic that the patient has unstable angina pectoris.

In another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard and decreased levels of high-density lipoprotein relevant to a reference standard in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, reduced heart rate relevant to a reference standard and an abnormal electrocardiogram relevant to a reference standard in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with decreased levels of high-density lipoprotein relevant to a reference standard and an abnormal electrocardiogram relevant to a reference standard in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when a history of angina when measured together with decreased levels of high-density lipoprotein relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with decreased levels of high-density lipoprotein relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with increased levels of an abnormal electrocardiogram and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with increased levels of an abnormal electrocardiogram relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate, decreased levels of high-density lipoprotein relevant to a reference standard and an abnormal electrocardiogram relevant to a reference standard in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard, decreased levels of high-density lipoprotein relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard, decreased levels of high-density lipoprotein relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard, an abnormal electrocardiogram relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard, an abnormal electrocardiogram relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard, a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard, an abnormal electrocardiogram relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, decreased levels of high-density lipoprotein relevant to a reference standard, an abnormal electrocardiogram relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, decreased levels of high-density lipoprotein relevant to a reference standard, a diagnosis of ischaemia, optionally by imaging, and a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, an abnormal electrocardiogram relevant to a reference standard, a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard, decreased levels of high-density lipoprotein relevant to a reference standard, and abnormal electrocardiogram relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard, decreased levels of high-density lipoprotein relevant to a reference standard, and abnormal electrocardiogram relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard, decreased levels of high-density lipoprotein relevant to a reference standard, a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard, an abnormal electrocardiogram relevant to a reference standard, a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, decreased levels of high-density lipoprotein relevant to a reference standard, an abnormal electrocardiogram relevant to a reference standard, a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample relative to the reference standard from the control population, when measured together with a history of angina, a reduced heart rate relevant to a reference standard, decreased levels of high-density lipoprotein relevant to a reference standard, an abnormal electrocardiogram relevant to a reference standard, a diagnosis of ischaemia, optionally by imaging, and a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina and a reduced heart rate relevant to a reference standard in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina and decreased levels of high-density lipoprotein in the patient relevant to a reference standard, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample measured relative to the reference standard from the control population, when measured together with a history of angina and an abnormal electrocardiogram relevant to a reference standard in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard and decreased levels of high-density lipoprotein relevant to a reference standard in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, reduced heart rate and an abnormal electrocardiogram in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels IGFBP-3 in the patient sample relative to the reference standard from the control population, when measured together with a history of angina, a reduced heart rate relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with decreased levels of high-density lipoprotein relevant to a reference standard and an abnormal electrocardiogram relevant to a reference standard in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with decreased levels of high-density lipoprotein relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with decreased levels of high-density lipoprotein relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with increased levels of an abnormal electrocardiogram relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with increased levels of an abnormal electrocardiogram relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, decreased levels of high-density lipoprotein relevant to a reference standard and an abnormal electrocardiogram relevant to a reference standard in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, decreased levels of high-density lipoprotein relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, decreased levels of high-density lipoprotein relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, an abnormal electrocardiogram relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, an abnormal electrocardiogram relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, a diagnosis of ischaemia, optionally by imaging, and dyslipidemia or a history of dyslipidemia in the patient, reflects that the patient has unstable angina pectoris.
(i) In yet another aspect of the present invention there is provided a method for diagnosing measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, an abnormal electrocardiogram relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, decreased levels of high-density lipoprotein relevant to a reference standard, an abnormal electrocardiogram relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, decreased levels of high-density lipoprotein relevant to a reference standard, a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, an abnormal electrocardiogram relevant to a reference standard, a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, decreased levels of high-density lipoprotein relevant to a reference standard, and abnormal electrocardiogram relevant to a reference standard and a diagnosis of ischaemia, optionally by imaging, in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, decreased levels of high-density lipoprotein relevant to a reference standard, and abnormal electrocardiogram relevant to a reference standard and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, decreased levels of high-density lipoprotein relevant to a reference standard, a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, an abnormal electrocardiogram relevant to a reference standard, a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, decreased levels of high-density lipoprotein relevant to a reference standard, an abnormal electrocardiogram relevant to a reference standard, a diagnosis of ischaemia, optionally by imaging, and dyslipidemia or a history of dyslipidemia in the patient, is diagnostic that the patient has unstable angina pectoris.

In yet another aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population, when measured together with a history of angina, a reduced heart rate relevant to a reference standard, decreased levels of high-density lipoprotein relevant to a reference standard, an abnormal electrocardiogram relevant to a reference standard, a diagnosis of ischaemia, optionally by imaging, and dyslipidemia or a history of dyslipidemia in the patient, is diagnostic that the patient has unstable angina pectoris.

In further aspect of the present invention there is provided a method for diagnosing inducible cardiac ischaemia in a patient, the method comprising:
(i) subjecting a patient suspected of having inducible ischaemia to a stress test;
(ii) measuring a peak heart rate level in the patient during the stress test;
(iii) measuring a resting heart rate level in the patient after the stress test;
(iv) determining a delta insulin-like growth factor binding protein 3 (ΔIGFBP-3) value which comprises:
   (a) determining the concentration of IGFBP-3 from a first sample obtained from the patient before the stress test;
   (b) determining the concentration of IGFBP-3 from a second sample obtained from the patient after the stress test;
   (c) determining the concentration difference in IGFBP-3 between the first and second samples to obtain a ΔIGFBP-3 value; and
   (d) comparing the ΔIGFBP-3 value from the patient sample against a reference standard of ΔIGFBP-3 from a control population,
wherein, where the patient has an increased heart rate level between the peak heart rate level (ii) and the resting heart rate level (iii) together with an increase in the ΔIGFBP-3 in the patient sample relative to the reference standard from the control population (iv), is diagnostic that the patient has inducible cardiac ischaemia.

In further aspect of the present invention there is provided a method for diagnosing inducible cardiac ischaemia in a patient, the method comprising:
(i) subjecting a patient suspected of having inducible ischaemia to a stress test;
(ii) measuring a peak heart rate level in the patient during the stress test;
(iii) determining if the patient has a transient ischaemia dilation;
(iv) measuring a resting heart rate level in the patient after the stress test;
(v) determining a delta insulin-like growth factor binding protein 3 (ΔIGFBP-3) value which comprises:
   (a) determining the concentration of IGFBP-3 from a first sample obtained from the patient before the stress test;
   (b) determining the concentration of IGFBP-3 from a second sample obtained from the patient after the stress test;
   (c) determining the concentration difference in IGFBP-3 between the first and second samples to obtain a ΔIGFBP-3 value; and
   (d) comparing the ΔIGFBP-3 value from the patient sample against a reference standard of ΔIGFBP-3 from a control population,
wherein, where the patient has confirmed transient ischaemia and an increase in heart rate between peak heart rate (ii) and resting heart rate (iv) together with an increase in the ΔIGFBP-3 in the patient sample relative to the reference standard from the control population (v), is diagnostic that the patient has inducible cardiac ischaemia.

In further aspect of the present invention there is provided a method for diagnosing inducible cardiac ischaemia in a patient, the method comprising:
(i) measuring a resting heart rate level in a patient suspected of having inducible ischaemia;
(ii) subjecting the patient to a stress test;
(iii) measuring a peak heart rate level in the patient during the stress test;
(iv) determining a delta insulin-like growth factor binding protein 3 (ΔIGFBP-3) value which comprises:
   (a) determining the concentration of IGFBP-3 from a first sample obtained from the patient before the stress test;
   (b) determining the concentration of IGFBP-3 from a second sample obtained from the patient after the stress test;
   (c) determining the concentration difference in IGFBP-3 between the first and second samples to obtain a ΔIGFBP-3 value; and
   (d) comparing the ΔIGFBP-3 value from the patient sample against a reference standard of ΔIGFBP-3 from a control population,
wherein, where the patient has a decreased heart rate level between the resting heart rate level (i) and the peak heart rate level (iii) together with an increase in the ΔIGFBP-3 in the patient sample relative to the reference standard from the control population (iv), is diagnostic that the patient has inducible cardiac ischaemia.

In further aspect of the present invention there is provided a method for diagnosing inducible cardiac ischaemia in a patient, the method comprising:
(i) measuring a resting heart rate level in a patient suspected of having inducible ischaemia;
(ii) subjecting the patient to a stress test;
(iii) determining if the patient has a transient ischaemia dilation;
(iv) measuring a resting heart rate level in the patient after the stress test;
(v) determining a delta insulin-like growth factor binding protein 3 (ΔIGFBP-3) value which comprises:
   (a) determining the concentration of IGFBP-3 from a first sample obtained from the patient before the stress test;
   (b) determining the concentration of IGFBP-3 from a second sample obtained from the patient after the stress test;
   (c) determining the concentration difference in IGFBP-3 between the first and second samples to obtain a ΔIGFBP-3 value; and
   (d) comparing the ΔIGFBP-3 value from the patient sample against a reference standard of ΔIGFBP-3 from a control population,
wherein, where the patient has a confirmed transient ischaemia dilation and a decreased heart rate level between the resting heart rate level (i) and the peak heart rate level (iv) together with an increase in the ΔIGFBP-3 in the patient sample relative to the reference standard from the control population (v), is diagnostic that the patient has inducible cardiac ischaemia.

In another aspect of the present invention there is provided a method for diagnosing an acute coronary syndrome in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the level of IGFBP-3 in the patient sample measured relative to the reference standard from the control population, when considered in combination with elevated levels of troponin, an abnormal electrocardiogram, a history of heart failure and/or a history of myocardial infarction, is diagnostic that the patient has an acute coronary syndrome.

In yet a further aspect of the present invention there is provided a method for diagnosing an acute coronary syndrome in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
wherein, an increase in the level of ΔIGFBP-3 in the patient sample measured relative to the reference standard from the control population, when considered in combination with elevated levels of troponin, an abnormal electrocardiogram, a history of heart failure and/or a history of myocardial infarction, is diagnostic that the patient has an acute coronary syndrome.

In a further aspect of the present invention there is provided a complex comprising IGFBP-3 bound to a binding agent which selectively binds to IGFBP-3.

In yet a further aspect of the present invention there is provided a peptide complex comprising IGFBP-3 bound to an antibody or antigen binding fragment thereof which selectively binds to IGFBP-3.

In another further aspect of the present invention there is provided a peptide complex comprising IGFBP-3 bound to a polyclonal antibody or antigen-binding fragment thereof which selectively binds to IGFBP-3.

In another further aspect of the present invention there is provided a peptide complex comprising IGFBP-3 bound to a monoclonal antibody or antigen-binding fragment thereof which selectively binds to IGFBP-3.

In yet another aspect of the present invention there is provided a complex comprising IGFBP-3 bound to an aptamer which selectively binds to IGFBP-3.

In yet a further aspect of the present invention there is provided a method for detecting a complex from a biological sample, wherein the complex comprises IGFBP-3 bound to a binding agent which selectively binds to IGFBP-3 and the method comprises detecting IGFBP-3 bound to the binding agent.

In yet a further aspect of the present invention there is provided a method for detecting a complex from a biological sample, wherein the complex comprises IGFBP-3 bound to a binding agent which selectively binds to IGFBP-3 and the method comprises detecting IGFBP-3 bound to the binding agent measured together with one or more clinical risk factors selected from a history of angina, a decreased heart rate relative to a reference standard, a decreased level of high-density lipoprotein relative to a reference standard, an abnormal electrocardiogram, a diagnosis of ischaemia, optionally by imaging, and dyslipidemia or a history of dyslipidemia.

In yet a further aspect of the present invention there is provided a method for detecting a peptide complex from a biological sample, wherein the peptide complex comprises IGFBP-3 bound to an antibody or antigen-binding fragment thereof which selectively binds to IGFBP-3, and the method comprises detecting IGFBP-3 bound to the antibody or antigen-binding fragment thereof.

In yet a further aspect of the present invention there is provided a method for detecting a peptide complex from a biological sample, wherein the peptide complex comprises IGFBP-3 bound to an antibody or antigen-binding fragment thereof which selectively binds to IGFBP-3, and the method comprises detecting IGFBP-3 bound to the antibody or antigen-binding fragment thereof measured together with one or more clinical risk factors selected from a history of angina, a decreased heart rate relative to a reference standard, a decreased level of high-density lipoprotein relative to a reference standard, an abnormal electrocardiogram, a diagnosis of ischaemia, optionally by imaging, and dyslipidemia or a history of dyslipidemia.

In yet a further aspect of the present invention there is provided a method for detecting a peptide complex from a biological sample, wherein the peptide complex comprises IGFBP-3 bound to a monoclonal antibody or antigen-binding fragment thereof which selectively binds to IGFBP-3, and the method comprises detecting IGFBP-3 bound to the monoclonal antibody or antigen-binding fragment thereof.

In yet a further aspect of the present invention there is provided a method for detecting a peptide complex from a biological sample, wherein the peptide complex comprises IGFBP-3 bound to a monoclonal antibody or antigen-binding fragment thereof which selectively binds to IGFBP-3, and the method comprises detecting IGFBP-3 bound to the monoclonal antibody or antigen-binding fragment thereof measured together with one or more clinical risk factors selected from a history of angina, a decreased heart rate relative to a reference standard, a decreased level of high-density lipoprotein relative to a reference standard, an abnormal electrocardiogram, a diagnosis of ischaemia, optionally by imaging, and dyslipidemia or a history of dyslipidemia.

In yet a further aspect of the present invention there is provided a method for detecting a peptide complex from a biological sample, wherein the peptide complex comprises IGFBP-3 bound to a polyclonal antibody or antigen-binding fragment thereof which selectively binds to IGFBP-3, and the method comprises detecting IGFBP-3 bound to the polyclonal antibody or antigen-binding fragment thereof.

In yet a further aspect of the present invention there is provided a method for detecting a peptide complex from a biological sample, wherein the peptide complex comprises IGFBP-3 bound to a polyclonal antibody or antigen-binding fragment thereof which selectively binds to IGFBP-3, and the method comprises detecting IGFBP-3 bound to the polyclonal antibody or antigen-binding fragment thereof measured together with one or more clinical risk factors selected from a history of angina, a decreased heart rate relative to a reference standard, a decreased level of high-density lipoprotein relative to a reference standard, an abnormal electrocardiogram, a diagnosis of ischaemia, optionally by imaging, and dyslipidemia or a history of dyslipidemia.

In yet a further aspect of the present invention there is provided a method for detecting a complex from a biological sample, wherein the complex comprises IGFBP-3 bound to an aptamer which selectively binds to IGFBP-3 and the method comprises detecting IGFBP-3 bound to the aptamer, and the method comprises detecting IGFBP-3 bound to the aptamer.

In yet a further aspect of the present invention there is provided a method for detecting a complex from a biological sample, wherein the complex comprises IGFBP-3 bound to an aptamer which selectively binds to IGFBP-3 and the method comprises detecting IGFBP-3 bound to the aptamer, and the method comprises detecting IGFBP-3 bound to the aptamer measured together with one or more clinical risk factors selected from a history of angina, a decreased heart rate relative to a reference standard, a decreased level of high-density lipoprotein relative to a reference standard, an abnormal electrocardiogram, a diagnosis of ischaemia, optionally by imaging, and dyslipidemia or a history of dyslipidemia.

In yet a further aspect of the present invention there is provided a binding agent which selectively binds to IGFBP-3.

In yet another aspect of the present invention there is provided an antibody or antigen-binding fragment which selectively binds to IGFBP-3.

In yet a further aspect of the present invention there is provided a monoclonal antibody, a polyclonal antibody, a chimeric antibody or a humanized antibody which selectively binds to IGFBP-3, or an antigen-binding fragment of a monoclonal, polyclonal, chimeric or humanized antibody which selectively binds to IGFBP-3.

In yet another aspect of the present invention there is provided a monoclonal antibody or antigen-binding fragment thereof which selectively binds to IGFBP-3.

In yet a further aspect of the present invention there is provided an antibody or antigen-binding fragment which selectively binds to IGFBP-3, which antibody or antigen-binding fragment comprises a detectable label.

In yet another aspect of the present invention there is provided an antibody or antigen-binding fragment which selectively binds to IGFBP-3, which antibody or antigen-binding fragment is immobilized on a substrate.

In yet a further aspect of the present invention there is provided a binding agent comprising a peptide framework comprising one or more complementarity determining regions derived from an antibody which selectively binds to IGFBP-3.

In yet a further aspect of the present invention there is provided a binding agent comprising a peptide framework comprising three complementarity determining regions derived from an antibody which selectively binds to IGFBP-3.

In yet another aspect of the present invention there is provided an aptamer or aptamer ligand binding domain which selectively binds to IGFBP-3.

In a further aspect of the present invention there is provided a test kit or article of manufacture for diagnosing unstable angina pectoris in a patient, the test kit or article of manufacture comprising a binding agent which selectively binds to IGFBP-3, and optionally, instructions for how to diagnose unstable angina pectoris in the patient.

In yet another aspect of the present invention there is provided a test kit or article of manufacture for diagnosing unstable angina pectoris in a patient, the test kit or article of manufacture comprising an antibody or aptamer which selectively binds to IGFBP-3, and optionally, instructions for how to diagnose unstable angina pectoris in the patient.

In yet a further aspect of the present invention there is provided a test kit or article of manufacture for diagnosing unstable angina pectoris in a patient, the test kit or article of manufacture comprising a monoclonal antibody or antigen-binding fragment thereof which selectively binds to IGFBP-3, and optionally, instructions for how to diagnose unstable angina pectoris in the patient.

### BRIEF DESCRIPTION OF THE FIGURES

FIG. 1 shows IGFBP-3 levels in acute chest pain patients (SPACE study, T=0 sample only). This patient cohort includes a total of n=1018 patients comprising: n=72 unstable angina pectoris; n=201 myocardial infarction; n=420 non-cardiac chest pain; n=275 undifferentiated chest pain; and n=50 other cardiac. IGFBP-3 levels (ng/mL) were measured by ELISA using the assay described in Example 1.

FIGS. 2(A)-2(C) show the Receiver Operating Curve (ROC) performance of IGFBP-3 for various non/cardiac diagnoses in the SPACE cohort. (A) Dx of myocardial infarction (MI): IGFBP-3 (AUC: 0.482±0.023); NT-proBNP (AUC: 0.647±0.022); and hsTnT (AUC: 0.931±0.010). (B) Dx of unstable angina pectoris (UAP): IGFBP-3 (AUC: 0.367±0.035); NT-proBNP (AUC: 0.581±0.033); and hsTnT (AUC: 0.407±0.031). Dx of non-cardiac chest pain (NCCP): IGFBP-3 (AUC: 0.581±0.018); NT-proBNP (AUC: 0.262±0.016); and hsTnT (AUC: 0.272±0.016).

FIG. 3 shows Receiver Operating Curve (ROC) performance involving the clinical variables of (i) AngHx+abnormal ECG (AUC=0.728±0.027), (ii) AngHx+abnormal ECG+IGFBP-3 (AUC=0.767±0.027), (iii) AngHx+abnormal ECG+HxDyslipidemia+IGFBP-3 (AUC=0.784±0.024) and (iv) AngHx+abnormal ECG+HxDyslipidemia+abnormal HDL+IGFBP-3 (AUC=0.797±0.024) for the diagnosis of unstable angina pectoris in the SPACE patient cohort. Levels of IGFBP-3 were measured at index presentation (i.e. T=0).

FIG. 4 shows Receiver Operating Curve (ROC) performance involving the clinical variables of (i) AngHx+abnormal ECG (AUC=0.716±0.031), (ii) AngHx+abnormal ECG+ΔIGFBP-3 (AUC=0.782±0.029), (iii) AngHx+abnormal ECG+abnormal HDL+ΔIGFBP-3 (AUC=0.802±0.027) for the diagnosis of unstable angina pectoris in the SPACE patient cohort.

FIG. 5 shows Receiver Operating Curve (ROC) performance involving the clinical variables of (i) hsTnT+HxHF+HxMI+abnormal ECG (AUC=0.802±0.024) and (ii) hsTnT+HxHF+HxMI+abnormal ECG+IGFBP-3 (AUC=0.840±0.020) and (iii) hsTnT+HxHF+HxMI+abnormal ECG+IGFBP-3+NT-proBNP (AUC=0.811±0.023) for the diagnosis of acute coronary syndromes in the SPACE patient cohort. Levels of IGFBP-3 were measured at index presentation (i.e. T=0).

FIGS. 6(A)-6(B) show the (A) 264 amino acid sequence of the IGFBP-3 propeptide (SEQ ID NO: 1) and (B) the 27 amino acid signal peptide sequence (SEQ ID NO: 2).

FIG. 7 shows Receiver Operating Curve (ROC) performance involving the clinical variables of (i) IGFBP-3 (AUC=0.638±0.050; black line), (ii) hsTnI (AUC=0.557±0.055; pale grey line), (iii) NT-proBNP (AUC=0.563±0.051; light grey line) and (iv) ΔGDF15 (AUC=0.564±0.054; mid-grey line) for the identification of ischemia in the BASEL VIII patient cohort following stress testing.

FIG. 8 shows Receiver Operating Curve (ROC) performance involving the clinical variables of (i) peak heart rate+resting heart rate (AUC=0.702±0.045), (ii) confirmed transient ischaemic dilation (TID)+peak heart rate+resting heart rate (AUC=0.725±0.045), (iii) confirmed TID+peak heart rate+resting heart rate+ΔIGFBP-3 (AUC=0.763±0.043) for the identification of ischemia in the BASEL VIII patient cohort following stress testing.

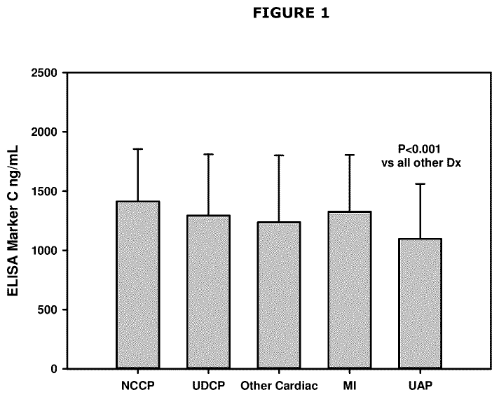

### DETAILED DESCRIPTION

### General Definitions

Unless specifically defined otherwise, all technical and scientific terms used herein shall be taken to have the same meaning as commonly understood by one of ordinary skill in the art to which the inventions belong (for example, in immunology, immunohistochemistry, protein chemistry, and biochemistry).

Unless otherwise indicated, the recombinant protein and immunological techniques utilized in the present invention are standard procedures well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al., (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

Any example or embodiment described herein shall be taken to apply mutatis mutandis to any other example or embodiment unless specifically stated otherwise.

### Selected Definitions

The terms “abnormal HDL” and “abnormal high-density lipoprotein” as used herein means an amount or concentration of HDL which is considered abnormal for a given age range. For example, abnormal HDL may include an amount or concentration that is abnormally high (e.g. >1.5 mM) or abnormally low (e.g. <1.03 mM). A person skilled in the art would understand how to make a routine assessment with respect to abnormal HDL by comparing the level of HDL from a test sample against a reference interval or value from a control population having normal HDL levels. The present invention is typically concerned with a decreased level of high-density lipoprotein relative to a reference standard.

The term “ACS” as used herein means acute coronary syndrome. Examples of acute coronary syndromes include, but are not limited to, unstable angina or unstable angina pectoris; cardiac ischemia and myocardial ischemia; Type 1 and Type 2 (acute) myocardial infarction including ST-elevation myocardial infarction and non-ST myocardial infarction; acute cardiac injury; acute cardiac damage resulting from acute drug toxicity, acute cardiomyopathies and cardiac transplant rejection.

The term “angina” as used herein means any form of chest pain whether that chest pain was experienced historically (e.g. “history of angina”) or in an acute setting.

For any avoidance of doubt the term “history of angina” is taken to mean a patient who has had any history of cardiovascular disease or has a history of chest pain complaints. The terms “history of angina”, “history of cardiovascular disease” and “history of chest pain” as used herein are therefore synonymous.

The term “antibody” refers to an immunoglobulin molecule capable of selectively binding to a target, such as IGFBP-3, by virtue of an antigen binding site contained within at least one variable region. This term includes four chain antibodies (e.g., two light chains and two heavy chains), recombinant or modified antibodies (e.g., chimeric antibodies, humanized antibodies, primatized antibodies, de-immunized antibodies, half antibodies, bispecific antibodies) and single domain antibodies such as domain antibodies and heavy chain only antibodies (e.g., camelid antibodies or cartilaginous fish immunoglobulin new antigen receptors (IgNARs)). An antibody generally comprises constant domains, which can be arranged into a constant region or constant fragment or fragment crystallisable (Fc). Preferred forms of antibodies comprise a four-chain structure as their basic unit. Full-length antibodies comprise two heavy chains (˜50-70 kDa) covalently linked and two light chains (˜23 kDa each). A light chain generally comprises a variable region and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH which is −330-440 amino acids in length). The light chain variable region is aligned with the variable region of the heavy chain. The antibody heavy chain can comprise 2 or more additional CH domains (such as, CH2, CH3 and the like) and can comprise a hinge region can be identified between the CH1 and Cm constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (preferably human) antibody. The term “antibody” encompasses not only intact polyclonal or monoclonal antibodies, but also variants, fusion proteins comprising an antibody portion with an antigen binding site, humanised antibodies, human antibodies, chimeric antibodies, primatised antibodies, de-immunised antibodies or veneered antibodies.

The term “antigen-binding fragment” or “antigen-binding antibody fragment” shall be taken to mean any fragment of an antibody that retains the ability to bind to Fibα and preferably one which specifically binds to IGFBP-3. This term includes a Fab fragment, a Fab′ fragment, a F(ab′) fragment, a single chain antibody (SCA or SCAB) amongst others. A “Fab fragment” consists of a monovalent antigen-binding fragment of an antibody molecule, and can be produced by digestion of a whole antibody molecule with the enzyme papain, to yield a fragment consisting of an intact light chain and a portion of a heavy chain. A “Fab′ fragment” of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fv fragment” is a genetically engineered fragment containing the variable region of a light chain and the variable region of a heavy chain expressed as two chains. A “single chain antibody” (SCA) is a genetically engineered single chain molecule containing the variable region of a light chain and the variable region of a heavy chain, linked by a suitable, flexible polypeptide linker.

The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass, as well as fragments of such antibodies, so long as they exhibit the desired biological activity (U.S. Pat. No. 4,816,567; and Morrison et al. (1984) Proc. Natl Acad. Sci USA 81:6851-6855).

The term “humanized antibody” shall be understood to refer to a chimeric molecule, generally prepared using recombinant techniques, having an epitope binding site derived from an immunoglobulin from a non-human species and the remaining immunoglobulin structure of the molecule based upon the structure and/or sequence of a human immunoglobulin. The antigen-binding site preferably comprises the complementarity determining regions (CDRs) from the non-human antibody grafted onto appropriate framework regions in the variable domains of human antibodies and the remaining regions from a human antibody. Epitope binding sites may be wild type or modified by one or more amino acid substitutions. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When non-human antibodies are prepared with respect to a particular epitope, the variable regions can be “reshaped” or “humanized” by grafting CDRs derived from non-human antibody on the FRs present in the human antibody to be modified.

The term “binding agent” as used herein is intended to refer to any molecule that binds (e.g.) IGFBP-3 including isoforms thereof, and the term binding agent includes small molecules, antibodies from any species whether polyclonal or monoclonal, antigen-binding fragments such as Fab and Fab2, humanized antibodies, chimeric antibodies, or antibodies modified in other ways including substitution of amino acids, and/or fusion with other peptides or proteins (e.g. PEG). It also includes receptors or binding proteins from any species or modified forms of them. In one example, the binding agent specifically binds to IGFBP-3, as well as isoforms and fragments thereof.

As used herein, the term “antigenic variant” refers to polypeptide sequences different from the specifically identified sequences, wherein one or more amino acid residues are deleted, substituted, or added. Substitutions, additions or deletions of 1, 2, 3 or 4 amino acids are specifically contemplated. Variants may be naturally-occurring allelic antigenic variants, or non-naturally occurring antigenic variants. Variants may be from the same or from other species and may encompass homologues, paralogues and orthologues. In certain embodiments, antigenic variants of the polypeptides useful in the invention have biological activities including hormone function or antigenic-binding properties that are the same or similar to those of the parent polypeptides. The term “antigenic variant” with reference to (poly)peptides encompasses all forms of polypeptides as defined herein. The term “antigenic variant” encompasses naturally occurring, as well as recombinant and synthetic produced polypeptides.

The term “AUC” means Area Under the Curve which yields information about the strength of a correlation determined by the Receiver Operating Curve analysis. Typical ROC values where the AUC>0.70 yields a statistically significant correlation.

The term “biological sample” as used herein includes biological fluids selected from blood including venous blood and arterial blood, plasma, serum, interstitial fluid, or any other body fluid. The term “biological sample” also includes heart tissue sample. The term “biological sample” and “body fluid sample” as used herein refers to a biological sample or a sample of bodily fluid obtained for the purpose of, for example, diagnosis, prognosis, classification or evaluation of a subject of interest, such as a patient. In certain embodiments, such a sample may be obtained for diagnosing a cardiac disorder, for performing risk stratification of a cardiac disorder, for making a prognosis of a disease course in a patient with a cardiac disorder, for identifying a patient with elevated risk of a cardiac disorder, or combinations thereof. In addition, a person skilled in the art would realise that certain body fluid samples would be more readily analysed following a fractionation or purification procedure, for example, separation of whole blood into serum or plasma components.

The term “comparing” has used herein has an ordinary meaning attached to it and is intended to mean a side-by-side comparison between the measured level of IGFBP-3 from (e.g.) a test sample and the measured level of IGFBP-3 from a control sample, such as that obtained from an individual or a population of individuals. In other examples, the level of IGFBP-3 measured from a test sample is compared to a test sample taken from an identical patient source at an earlier time point(s).

The terms “control population” and “suitable control population” according to the present invention refers to the mean circulating IGFBP-3 levels from sex- and age-matched subjects for which their cardiac disease or disorder status is known. The control population is used to provide a suitable reference interval by which a measured IGFBP-3 protein or isoform level is compared.

The term “dyslipidemia” as used herein means a disorder of lipoprotein metabolism, usually manifested as an elevation of plasma cholesterol and/or triglycerides, or a low/high HDL cholesterol level that contributes to the development of atherosclerosis. A diagnosis of dyslipidemia is made by measuring plasma levels of total cholesterol, triglycerides, and individual lipoproteins [13].

The term “Dx” as used herein means diagnosis or diagnostic.

The term “ECG” as used herein means electrocardiogram. In the context of the present invention an “abnormal ECG” is intended to mean an ECG in which the P, Q, R, S and/or T peaks display an abnormal pattern.

The term “effective amount” as used herein refers to the amount of a therapy that is sufficient to result in the prevention of the development, recurrence, or onset of a disease or condition and one or more symptoms thereof, to enhance or improve the prophylactic effect(s) of another therapy, reduce the severity, the duration of disease, ameliorate one or more symptoms of the disease or condition, prevent the advancement of the disease or condition, cause regression of the disease or condition, and/or enhance or improve the therapeutic effect(s) of another therapy.

The term “ELISA” as used herein means enzyme-linked immunosorbent assay.

The term “epitope” includes any antigenic (e.g., a protein) determinant capable of specific binding to an antibody. Epitope determinants usually consist of chemically active surface groupings of molecules such as amino acids or sugar side chains, and usually have specific three-dimensional structural characteristics, as well as specific charge characteristics. An epitope typically includes, for example, at least 3, 5 or 8-10 amino acids. The amino acids may be contiguous, or non-contiguous amino acids juxtaposed by tertiary folding. Conformational and non-conformational epitopes are distinguished in that the binding to the former but not the latter is lost in the presence of denaturing solvents.

The term “hsTnT” as used herein means high sensitivity Troponin T, and includes high sensitivity cardiac Troponin T (i.e. hscTnT).

The term “Hx” as used herein means history, for example “Hx angina” means history of angina.

The terms “HxHF” or “HFHx” as used herein mean a patient who has a history of heart failure.

The terms “HxMI” or “MIHx” as used herein mean a patient who has a history of myocardial infarction.

The terms “HxDyslipidemia”, “DyslipidemiaHx”, “HxDyslipid.” and “DyslipidHx” as used herein mean a history of dyslipidemia.

The term “IGFBP-3” as used herein means insulin-like growth factor binding protein 3. This includes, without limitation, a protein defined by SEQ ID NO: 1 (264 amino acid residues) as well as isoforms of IGFBP-3 and fragments of IGFBP-3. Insulin-like growth factor binding protein 3 is also known the art by the aliases “BP-53”, “IBP3” and “IBP-3” which terms may be used interchangeably throughout this specification with IGFBP-3.

The terms “delta-IGFBP-3” or “ΔIGFBP-3” as used herein mean the difference in IGFBP-3 levels measured in (i) a first patient sample taken at presentation (e.g. to a hospital emergency department or clinic) with a complaint of chest pain (i.e. T=0) and (ii) a second sample taken from the same patient at a later point in time which includes, by way of illustration only, 0.5 h, 1 h, 1.5 h, 2 h, 2.5 h, 3 h, 3.5 h, 4 h, 4.5 h, 5 h, 5.5 h, 6 h, 6.5 h, 7 h, 7.5 h, 8 h, 8.5 h, 9 h, 9.5 h and 10 h. For any avoidance of doubt, the terms “ABP-53”, “AIBP3” and “ΔIBP-3” may be taken mutandis mutatis with “ΔIGFBP-3” based on the definitions given above. Further, the person skilled in the art would recognise that the terms “ΔIGFBP-3”, “ΔBP-53”, “ΔIBP3” and “ΔIBP-3” apply to the determination of a change in the IGFBP-3 levels either at presentation to (e.g.) to a hospital or clinic or at any later time point following a complaint of chest pain. In other words, the terms “ΔIGFBP-3”, “ΔBP-53”, “ΔIBP3” and “ΔIBP-3” also encompasses repeat assays performed over multiple time points.

An “increase” or “decrease” in the level of IGFBP-3 (or any other biomarker for that matter) compared with a control, or a “change” or “deviation” from a control (level) in one example is statistically significant. An increased level, decreased level, deviation from, or change from a control level or mean or historical control level can be considered to exist if the level differs from the control level by about 5% or more, by about 10% or more, by about 20% or more, or by about 50% or more compared to the control level. Statistically significant may alternatively be calculated as P<0.05. Increased levels, decreased levels, deviation, and changes can also be determined by recourse to assay reference limits or reference intervals. These can be calculated from intuitive assessment or non-parametric methods. Overall, these methods may calculate the 0.025, and 0.975 fractiles as 0.025*(n+1) and 0.975 (n+1). Such methods are well known in the art. Presence of a marker absent in a control may be seen as a higher level, deviation or change. Absence of a marker present in a control may be seen as a lower level, deviation or change.

The term “index presentation” as used herein means the point at which a patient presents to (e.g.) an emergency department, a clinic, a hospital, a surgery, a doctor's practice, a doctor or any other relevant medical forum, and information about the cardiac status of the patient is measured, including the patient's IGFBP-3 levels. For any avoidance of doubt, the term “index presentation” also includes determining the levels of IGFBP-3 in a patient or subject who has a new or recurring complaint of chest pain.

The term “isolated” as applied to the polypeptide sequences disclosed herein is used to refer to sequences that are removed from their natural cellular or other naturally-occurring biological environment. An isolated molecule may be obtained by any method or combination of methods including biochemical, recombinant, and synthetic techniques. The polypeptide sequences may be prepared by at least one purification step.

The term “level” as used herein is intended to refer to the amount per weight or weight per weight of an analyte of interest, (e.g.) IGFBP-3. It is also intended to encompass “concentration” expressed as amount per volume or weight per volume. The term “circulating level” is intended to refer to the amount per weight or weight per weight or concentration of, for example, IGFBP-3 present in the circulating fluid such as plasma, serum or whole blood.

As used herein, the terms “manage”, “managing”, and “management” in the context of the administration of a therapy to a subject refer to the beneficial effects that a subject derives from a therapy (e.g., a prophylactic or therapeutic agent) or a combination of therapies, while not resulting in a cure of the disease or condition. In certain examples, a subject is administered one or more therapies (e.g., one or more prophylactic or therapeutic agents) to “manage” the disease or condition so as to prevent the progression or worsening of the disease or condition.

The term “MACE” as used herein means major acute cardiac event.

The terms “marker” or “biomarker” in the context of an analyte means any antigen, molecule or other chemical or biological entity that is specifically found in circulation or associated with a particular tissue (e.g. heart muscle) that it is desired to be identified in or on a particular tissue affected by a disease or disorder, for example unstable angina. In specific examples, the marker is a circulating peptide (e.g.) IGFBP-3.

The terms “MI” and “AMI” as used herein mean (acute) myocardial infarction, a type of acute coronary syndrome.

The term “NCCP” as used herein means non-cardiac chest pain.

The term “NT-proBNP” as used herein means N-Terminal pro B-Type Natriuretic Peptide.

The terms “peptide” and “polypeptide” or “selectively binds” may be used interchangeably throughout this specification, and encompass amino acid chains of any length, including full length sequences in which amino acid residues are linked by covalent peptide bonds. Polypeptides useful in the present invention may be purified natural products, or may be produced partially or wholly using recombinant or synthetic techniques. The term “polypeptide” may refer to a polypeptide, an aggregate of a polypeptide such as a dimer or other multimer, a fusion polypeptide, a polypeptide fragment, a polypeptide variant, or derivative thereof. Polypeptides herein may have chain lengths of at least 40 amino acids, at least 50 amino acids, or at least 60, at least 70, at least 80, at least 90, at least 100, at least 110, at least 120, at least 130, at least 140, at least 150, at least 160, at least 170, at least 180, at least 190, at least 200, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250, at least 251, at least 252, at least 253, at least 254, at least 255, at least 256, at least 257, at least 258, at least 259, at least 260, at least 261, at least 262, at least 263, at least 264 amino acids. Reference to other polypeptides of the invention or other polypeptides described herein should be similarly understood.

The term “purified” as used herein does not require absolute purity. Purified refers in various embodiments, for example, to at least about 80%, 85%, 90%, 95%, 98%, or 99% homogeneity of a polypeptide, for example, in a sample. The term should be similarly understood in relation to other molecules and constructs described herein.

The term “Px” as used herein means prediction or prognostic.

Specifically, the term “reference interval” or “reference standard” as used herein is intended to refer to a figure within a statistical band of a representative concentration or alternatively a figure with an upper or lower concentration. The reference interval or reference standard will typically be obtained from subjects that do not have any pre-existing conditions that could result in artificially elevating the level of circulating IGFBP-3.

The term “ROC” means Receiver Operating Curve and a ROC plot depicts the overlap between two distributions by plotting the sensitivity versus 1-specificity for a complete range of decision thresholds.

The term “subject” or “patient” may be used interchangeably in this specification and it intended to refer to a human or non-human primate. In one example, the subject or patient is a human.

The terms “specifically binds” or “selectively binds” may be used interchangeably throughout this specification, and shall be taken to mean that the binding agent reacts or associates more frequently, more rapidly, with greater duration and/or with greater affinity to a particular substance than it does with alternative substances. For example, a binding agent that specifically binds to IGFBP-3, as well as isoforms thereof, or an epitope or immunogenic fragment thereof with greater affinity, avidity, more readily, and/or with greater duration than it binds to unrelated protein and/or epitopes or immunogenic fragments thereof. It is also understood by reading this definition that, for example, a binding agent that specifically binds to a first target (e.g. IGFBP-3) may or may not specifically bind to a second target. As such, “specific binding” does not necessarily require exclusive binding or non-detectable binding of another molecule. Generally, but not necessarily, reference to binding means specific binding.

In addition to computer/database methods known in the art, polypeptide antigenic variants may be identified by physical methods known in the art, for example, by screening expression libraries using antibodies raised against polypeptides of the invention (Sambrook et al., Molecular Cloning: A Laboratory Manual, 2nd Ed. Cold Spring Harbor Press, 1987) by recombinant DNA techniques also described by Sambrook et al. or by identifying polypeptides from natural sources with the aid of such antibodies.

As used herein, the term “therapeutic agent” refers to any molecule, compound, and/or substance that is used for the purpose of treating and/or managing a disease or disorder, such as unstable angina. Examples of therapeutic agents include, but are not limited to, proteins, immunoglobulins (e.g., multi-specific Igs, single chain Igs, Ig fragments, polyclonal antibodies and their fragments, monoclonal antibodies and their fragments), peptides (e.g., peptide receptors, selectins), binding proteins, biologics, proliferation-based therapy agents, hormonal agents, radioimmunotherapies, targeted agents, epigenetic therapies, differentiation therapies, biological agents, and small molecule drugs.

As used herein, the terms “therapies” and “therapy” can refer to any method(s), composition(s), and/or agent(s) that can be used in the prevention, treatment and/or management of a disease or condition or one or more symptoms thereof.

The term “TID” as used herein mean transient ischaemia dilation, which may be confirmed, for example, using spectral imaging or ultrasound.

The terms “TnT” and “cTnT” means Troponin T, typically derived from a cardiac source.

As used herein, the terms “treat”, “treatment” and “treating” in the context of the administration of a therapy to a subject refer to the reduction, inhibition, elimination or amelioration of the progression and/or duration of (e.g.) unstable angina, the reduction, inhibition, elimination or amelioration of the severity of (e.g.) unstable angina, and/or the amelioration of one or more symptoms thereof resulting from the administration of one or more therapies.

As used in this specification, the term “fragment” or “functional derivative” in relation to a polypeptide is a subsequence of a polypeptide that may be detected using a binding agent. The term may refer to a polypeptide, an aggregate of a polypeptide such as a dimer or multimer, a fusion polypeptide, a polypeptide fragment, a polypeptide variant or derivative thereof.

The term “UA” and “UAP” as used herein means unstable angina or unstable angina pectoris, a type of acute coronary syndrome.

The term “UDCP” as used herein means undifferentiated chest pain.

Term “variant” as used herein refers to polypeptide sequences different from the specifically identified sequences, wherein 1 to 6 or more or amino acid residues are deleted, substituted, or added. Substitutions, additions or deletions of one, two, three, four, five or six amino acids are contemplated. Variants may be naturally occurring allelic variants, or non-naturally occurring variants. Variants may be from the same or from other species and may encompass homologues, paralogues and orthologues. In certain embodiments, variants of the polypeptides useful in the invention have biological activities including signal peptide activity or antigenic-binding properties that are the same or similar to those of the parent polypeptides. The term “variant” with reference to polypeptides encompasses all forms of polypeptides as defined herein.

Variant polypeptide sequences exhibit at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to a sequence of the present invention. With regard to polypeptides, identity is found over a comparison window of at least 233 to 291 amino acid positions.

Polypeptide variants also encompass those which exhibit a similarity to one or more of the specifically identified sequences that is likely to preserve the functional equivalence of those sequences, including those which could not reasonably be expected to have occurred by random chance.

Polypeptide sequence identity and similarity can be determined in the following manner. The subject polypeptide sequence is compared to a candidate polypeptide sequence using BLASTP (from the BLAST suite of programs, version 2.2.18 [April 2008]]) in bl2seq, which is publicly available from NCBI (ftp://ftp.ncbi.nih.gov/blast/). The default parameters of bl2seq are utilized except that filtering of low complexity regions should be turned off.

The similarity of polypeptide sequences may be examined using the following UNIX command line parameters: bl2seq-i peptideseq1-j peptideseq2-F F-p blastp. The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match. Variant polypeptide sequences commonly exhibit an E value of less than 1×10⁻⁵, less than 1×10⁻⁶, less than 1×10⁻⁹, less than 1×10⁻¹², less than 1×10⁻¹⁵, less than 1×10⁻¹⁸ or less than 1×10⁻²¹ when compared with any one of the specifically identified sequences. Polypeptide sequence identity may also be calculated over the entire length of the overlap between a candidate and subject polypeptide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235) as discussed above are also suitable global sequence alignment programs for calculating polypeptide sequence identity. Use of BLASTP is preferred for use in the determination of polypeptide variants according to the present invention.

### DETAILED DESCRIPTION

Insulin-like growth factor binding protein 3 (IGFBP-3) is a 264 amino acid proprotein that binds insulin-like growth factors 1 and 2 (IGF-I/IGF-II). IGFBP-3 is glycosylated and phosphorylated and has a final molecular weight in the range 40-43 kDa [8].

The main function of IGFBP-3 appears to be in insulin growth factor (IGF) transport in the circulation as a 150 kDa complex of IGFBP-3/IGF-I or IGF-II and a third factor known as acid labile subunit (ALS). Approximately 80%+circulating of IGF-I/IGF-II is bound to IGFBP-3 and thus, IGFBP-3 plays a major role in IGF bioavailability.

IGFBP-3 is produced in multiple cell types including the liver, kidney, gut, uterus and placenta and the major regulators of its production are growth-hormone (GH), nutrition and age. Because of its binding to IGF and its dependence upon growth-hormone, IGFBP-3 is measured clinically as a surrogate of disorders of growth-hormone secretion or action, particularly in relation to pediatric growth.

Functionally, two unrelated IGFBP-3 receptors have been described. First, the low-density lipoprotein receptor-related protein 1 (LRP1) [8] and second, the transmembrane protein TMEM219 [9]. It has also been proposed that circulating IGFBP-3 can enter cells by both clathrin-mediated and caveolin-mediated endocytosis [10] possibly involving the transferrin receptor [11].

There is limited information about IGFBP-3 in the context of acute cardiac conditions such as ischemia or chest pain.

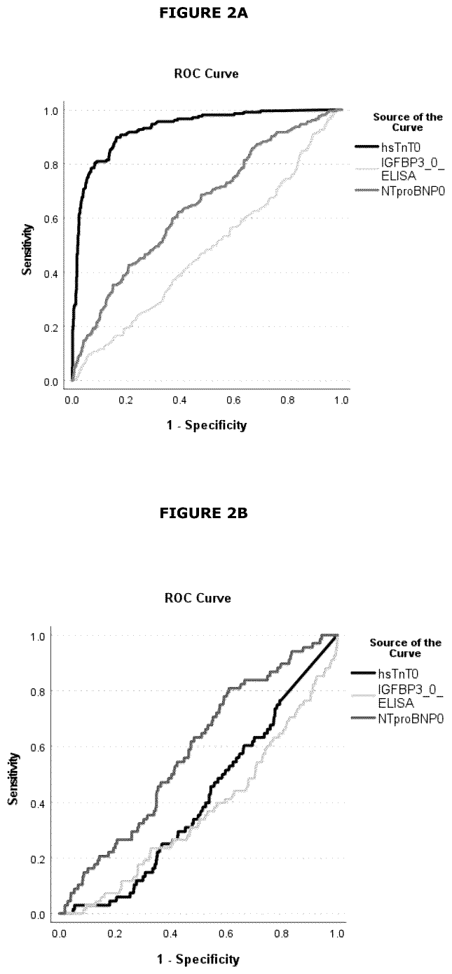

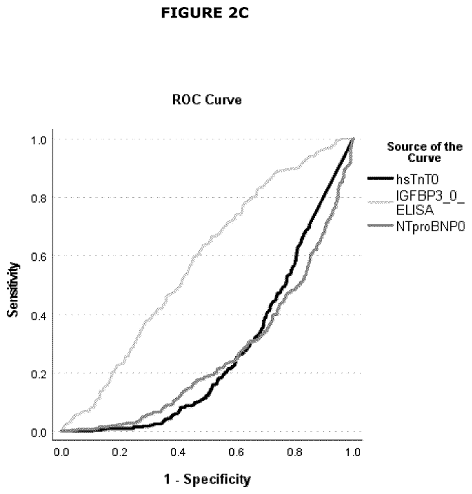

Indeed, the limited clinical utility of IGFBP-3 as a stand-alone biomarker of acute coronary syndromes is evident when the data presented in Example 2 is considered in conjunction with FIG. 2. Receiver Operating Curve (ROC) data for IGFBP-3 relative to NT-proBNP and hsTnT for the diagnosis of myocardial infarction (MI), unstable angina pectoris (UAP) and non-cardiac chest pain (NCCP) reveals that IGFBP-3 performs very poorly. By way of illustration, the area under the curve (AUC) for IGFBP-3 of AUC=0.482±0.023 indicates it has no clinical utility for the diagnosis of myocardial infarction relative the “gold standard” high-sensitivity Troponin T with an AUC=0.931±0.010. Refer to Table 1 and FIG. 2A.

However, the information presented in the Examples 3-6 which follow demonstrate a bone fide clinical utility for IGFBP-3 in the identification of subjects who have unstable angina. This is significant because there is no current biomarker used in a hospital/clinic setting to triage patients with unstable angina from those who are afflicted by other cardiac or non-cardiac conditions including, for example, (acute) myocardial infarction or heart failure.

The data presented in Example 3 reveals that patients with clinically adjudicated unstable angina from within the “SPACE” cohort (n=72; refer to Example 2) demonstrated decreased levels of IGFBP-3 relative to other diagnoses, including myocardial infarction (n=201), non-cardiac chest pain (n=420), undifferentiated chest pain (n=275), and other cardiac conditions (n=50).

Specifically, with reference to Table 2 and FIG. 2B, IGFBP-3 displayed superior performance at index presentation for the diagnosis of UAP compared to NT-proBNP and hsTnT, with AUC values of 0.367±0.035, 0.581±0.033 and 0.407±0.031, respectively. Refer to Table 2 and FIG. 2B.

Accordingly, in an aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the levels of IGFBP-3 in the patient sample relative to the reference standard from the control population is diagnostic that the patient has unstable angina pectoris.

More importantly, when IGFBP-3 is considered alongside other risk factors including (e.g.) a history of angina, a decreased heart rate relative to a reference standard, a decreased level of high-density lipoprotein relative to a reference standard, an abnormal electrocardiogram, a diagnosis of ischaemia, optionally by imaging, and dyslipidemia or a history of dyslipidemia, these data reveal a significant improvement in the diagnostic performance of the various biomarker panels for UAP.

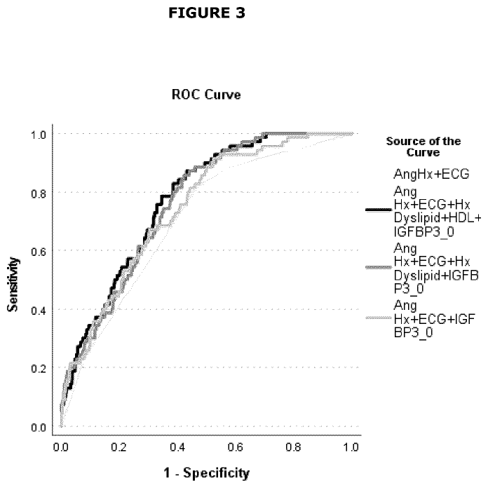

For example, in reference to Table 6 in Example 3, when read in conjunction with FIG. 3, the AUC increases from 0.728±0.027 to 0.767±0.027 when IGFBP-3 is added to history of angina (HxAng) and abnormal ECG.

Performance of the assay was further improved when the additional risk factors of dyslipidemia and/or abnormal HDL are taken into consideration. Specifically, the AUC for the diagnosis for UAP increases from 0.767±0.027 to 0.784±0.024 on the addition of dyslipidemia, with a further improvement to AUC=0.797±0.024 when abnormal HDL is also added to the biomarker panel.

In one example according to these and other aspects, a decreased level of high-density lipoprotein relative to a reference standard includes a decreased level of high-density lipoprotein C.

Given the log scale associated with Receiver Operating Curves, a person skilled in the art would recognise that any increase in the area under the curve beyond AUC=0.728±0.027 (i.e. based on HxAng+ECG) by including one or more of IGFBP-3, HxDyslipidemia and/or abnormal HDL, represents a statistically significant improvement for the diagnosis of unstable angina pectoris.

Accordingly, in one aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the level of IGFBP-3 in the patient sample measured relative to the reference standard from the control population, when considered in combination with history of angina and an abnormal electrocardiogram, is diagnostic that the patient has unstable angina pectoris.

In an example according to this aspect of the present invention, the method further comprises an assessment of additional risk factors comprising dyslipidemia, a history of dyslipidemia and/or abnormal levels of high-density lipoprotein (HDL).

Accordingly, in a separate aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the level of IGFBP-3 in the patient sample measured relative to the reference standard from the control population, when considered in combination with history of angina, an abnormal electrocardiogram, an increased heart rate, dyslipidemia and history of dyslipidemia, is diagnostic that the patient has unstable angina pectoris.

In further separate aspect of the present invention there is provided a method for diagnosing unstable angina pectoris in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, a decrease in the level of IGFBP-3 in the patient sample measured relative to the reference standard from the control population, when considered in combination with history of angina, an abnormal electrocardiogram, an increased heart rate, dyslipidemia, history of dyslipidemia, and abnormal levels of high-density lipoprotein, is diagnostic that the patient has unstable angina pectoris.

The data referred to above provides the first evidence as to the utility of IGFBP-3 in determining unstable angina pectoris.

Unexpectedly, however, when IGFBP-3 was measured in combination with other risk factor(s), further enhancements in assay specificity and sensitivity were achieved when the levels of IGFBP-3 are interrogated over a ˜0.5-10 h window following presentation with a complaint of chest pain (referred to herein after as “delta-IGFBP-3” or “ΔIGFBP-3”).

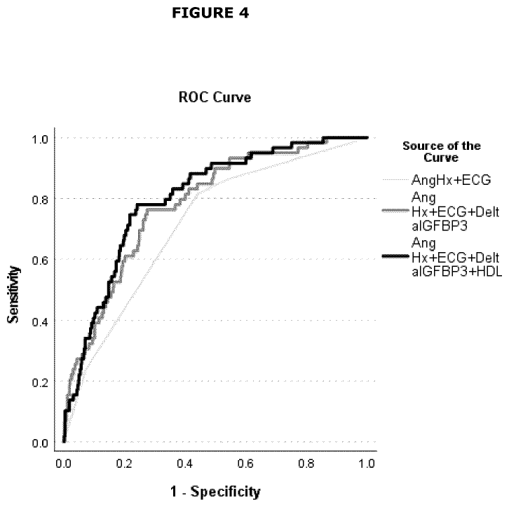

Specifically, the data presented in Example 4, read in conjunction with FIG. 4, demonstrates the power of this interrogation when the model includes a ΔIGFBP-3 value as determined across a two hour window from an initial complaint of chest pain including (e.g.) at index presentation. In terms of the ROC data, there is a marked increase in the diagnostic power from AUC=0.716±0.031 to AUC=0.782±0.029 when ΔIGFBP-3 is added to HxAng and abnormal ECG. Further (significant) improvements in the diagnostic power of the assay are achieved when abnormal HDL levels are added to HxAng, abnormal ECG and ΔIGFBP-3 with an AUC=0.802±0.027. Refer to Table 10 in Example 4.

Accordingly, in yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
wherein, an increase in the ΔIGFBP-3 value in the patient sample measured relative to the reference standard from the control population is diagnostic that the patient has unstable angina pectoris.

In yet another aspect of the present invention there is provided a method for diagnosing unstable angina pectoris in a patient, the method comprising:
(i) determining a delta insulin-like growth factor binding protein 3 (ΔIGFBP-3) value which comprises:
   (a) determining the concentration of IGFBP-3 from a first sample obtained from the patient;
   (b) determining the concentration of IGFBP-3 from a second sample obtained from the patient at a time point after (a); and
   (c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population,
   wherein, an increase in the ΔIGFBP-3 in the patient sample measured relative to the reference standard from the control population, when measured together with a history of angina and an abnormal electrocardiogram relevant to a reference standard in the patient, is diagnostic that the patient has unstable angina pectoris.

In an example according to any of the methods described herein, the concentration or level of IGFBP-3 is obtained from a sample taken from a patient at index presentation. This includes, without limitation, at a time when a patient (e.g.) presents to an emergency department, a clinic, a hospital, a surgery, a doctor's practice, a doctor or any other relevant medical forum, and information about the cardiac status of the patient is measured, including the patient's IGFBP-3 levels, or and a time when a patient identifies or presents with a complaint of chest pain during the hospital admission period or during a follow-up consultation.

In another example according to these and other aspects, the time point between obtaining a first sample (a) and obtaining a second sample (b) from the patient is typically between about half an hour to about 10 hours. This includes, without limitation, about 0.5 h, about 1 h, about 1.5 h, about 2 h, about 2.5 h, about 3 h, about 3.5 h, about 4 h, about 4.5 h, about 5 h, about 5.5 h, about 6 h, about 6.5 h, about 7 h, about 7.5 h, about 8 h, about 8.5 h, about 9 h, about 9.5 h or about 10 h.

In a further example, the time point between obtaining a first sample (a) and obtaining a second sample (b) from the patient is typically about 2 h.

The levels of ΔIGFBP-3 determined according to the methods described and claimed herein may be further considered in conjunction with other risk factors selected from an increased heart rate relative to a reference standard, a decreased level of high-density lipoprotein relative to a reference standard, a diagnosis of ischaemia, a diagnosis of ischaemia, optionally by imaging, and dyslipidemia or a history of dyslipidemia.

A person skilled in the art would appreciate the terms “measured together with” or “measured together with one or more risk factors” in the context of (e.g.) a history of angina, an increased heart rate relevant to a reference standard, an abnormal electrocardiogram, a decreased level of high-density lipoprotein relevant to a reference standard, a diagnosis of ischaemia, optionally by imaging, or dyslipidemia or a history of dyslipidemia, in isolation or in any combination, is intended to mean that (i) the patient has presented with one or more of a history of angina, an increased heart rate relevant to a reference standard, an abnormal electrocardiogram, a decreased level of high-density lipoprotein relevant to a reference standard, a diagnosis of ischaemia, optionally by imaging or dyslipidemia or a history of dyslipidemia or (ii) alongside an interrogation of the IGFBP-3 or ΔIGFBP-3 levels in a sample obtained from the patient, it is established that the patient has one or more of a history of angina, an increased heart rate relevant to a reference standard, an abnormal electrocardiogram, a decreased level of high-density lipoprotein relevant to a reference standard, a diagnosis of ischaemia, optionally by imaging, or dyslipidemia or a history of dyslipidemia.

Accordingly, in any of the methods disclosed herein for diagnosing unstable angina pectoris in a patient, the patient may optionally present with one or more of the following features:
(i) history of unstable angina [feature (i)];
(ii) an abnormal electrocardiogram [feature (ii)];
(iii) a reduced heart rate relevant to a reference standard [feature (iii)];
(iv) a decreased level of high-density lipoprotein relevant to a reference standard [feature (iv)];
(v) a diagnosis of ischaemia, optionally by imaging [feature (v)]; and
(vi) dyslipidemia or a history of dyslipidemia [feature (vi)].

In a related example, and according to the definitions provided above, the patient with unstable angina presents with:
(A) feature (i);
(B) features (i) and (ii);
(C) features (i) and (iii);
(D) features (i) and (iv);
(E) features (i) and (v);
(F) features (i) and (vi);
(G) features (i), (ii) and (iii);
(H) features (i), (ii) and (iv);
(I) features (i), (ii) and (v);
(J) features (i), (ii) and (vi);
(K) features (i), (iii) and (iv);
(L) features (i), (iii) and (v);
(M) features (i), (iii) and (vi);
(N) features (i), (iv) and (v);
(O) features (i), (iv) and (vi);
(P) features (i), (v) and (vi);
(Q) features (i), (ii), (iii) and (iv);
(R) features (i), (ii), (iii) and (v);
(S) features (i), (ii), (iii) and (vi);
(T) features (i), (ii), (iv) and (v);
(U) features (i), (ii), (iv) and (vi);
(V) features (i), (ii), (v) and (vi);
(W) features (i), (iii), (iv) and (v);
(X) features (i), (iii), (iv) and (vi);
(Y) features (i), (iii), (v) and (vi);
(Z) features (i), (vi), (v) and (vi);
(AA) features (i), (ii), (iii), (iv) and (v);
(BB) features (i), (ii), (iii), (iv) and (vi);
(CC) features (i), (ii), (iii), (v) and (vi);
(DD) features (i), (ii), (iv), (v) and (vi);
(EE) features (i), (iii), (iv), (v) and (vi); or
(FF) features (i). (ii), (iii), (iv), (v) and (vi).

Similarly, a person skilled in the art would appreciate the terms “when considered in combination with elevated levels of troponin, an abnormal electrocardiogram, a history of heart failure and/or a history of myocardial infarction” in the context of (e.g.) a diagnosis of an acute coronary syndrome is intended to mean that (i) the patient has presented with one or more of an elevated level of troponin, an abnormal electrocardiogram, a history of heart failure and/or a history of myocardial infarction or (ii) alongside an interrogation of the IGFBP-3 or ΔIGFBP-3 levels in a sample obtained from the patient, it is established that the patient has one or more of an elevated level of troponin, an abnormal electrocardiogram, a history of heart failure and/or a history of myocardial infarction.

Accordingly, in any of the methods disclosed herein for diagnosing an acute coronary syndrome in a patient, the patient may optionally present with one or more of the following features:
(vii) an elevated level of troponin [feature (vii)];
(viii) an abnormal electrocardiogram [feature (viii)];
(ix) a history of heart failure [feature (ix)];
(x) history of myocardial infarction [feature (x)].

In a related example, and according to the definitions provided above, the patient with unstable angina presents with:
(GG) feature (vii) and feature (viii);
(HH) feature (vii) and feature (ix);
(II) feature (vii) and feature (x);
(JJ) feature (viii) and feature (ix);
(KK) feature (viii) and feature (x);
(LL) feature (ix) and feature (x);
(MM) feature (vii), feature (viii) and feature (ix);
(NN) feature (vii), feature (viii) and feature (x);
(OO) feature (vii), feature (ix) and feature (x);
(PP) feature (viii), feature (ix) and feature (x); and
(QQ) feature (vii), feature (viii), feature (ix) and feature (x).

In the context of ischaemia short of infarction (e.g. caused by unstable angina pectoris), the diagnostic utility of IGFBP-3 is further confirmed by the data presented in Example 6. Here, the levels of IGFBP-3 were interrogated in patients recruited to a separate cohort (“Basel VIII”) as a biomarker of inducible ischaemia.

These data show that IGFBP-3 in conjunction with clinical risk factors of heart rate (i.e. peak and resting), as well as imaging confirming transient ischaemia dilation, may be used to develop a model to identify inducible ischaemia in the patient following stress testing. A determination of inducible ischaemia reflects that the patient has, or is predisposed to having, unstable angina pectoris angina. In turn, this information provides valuable information to a clinician in terms of further triage options and/or treatment regimes.

Accordingly, in another aspect of the present invention there is provided a method for diagnosing inducible ischaemia in a patient, the method comprising:
(i) subjecting a patient suspected of having inducible ischaemia to a stress test;
(ii) measuring a peak heart rate level in the patient during the stress test;
(iii) measuring a resting heart rate level in the patient after the stress test;
(iv) determining a delta insulin-like growth factor binding protein 3 (ΔIGFBP-3) value which comprises:
   (a) determining the concentration of IGFBP-3 from a first sample obtained from the patient before the stress test;
   (b) determining the concentration of IGFBP-3 from a second sample obtained from the patient after the stress test;
   (c) determining the concentration difference in IGFBP-3 between the first and second samples to obtain a ΔIGFBP-3 value; and
   (d) comparing the ΔIGFBP-3 value from the patient sample against a reference standard of ΔIGFBP-3 from a control population,
wherein, where the patient has an increased heart rate level between the peak heart rate level (ii) and the resting heart rate level (iii) together with an increase in the ΔIGFBP-3 in the patient sample relative to the reference standard from the control population (iv), is diagnostic that the patient has inducible ischaemia.

In a related aspect, the present invention provides a method for diagnosing inducible ischaemia in a patient, typically a patient suspected of having inducible ischaemia, the method comprising:
determining a delta insulin-like growth factor binding protein 3 (ΔIGFBP-3) value which comprises:
   (a) determining the concentration of IGFBP-3 from a first sample obtained from the patient before a stress test;
   (b) determining the concentration of IGFBP-3 from a second sample obtained from the patient after the stress test;
   (c) determining the concentration difference in IGFBP-3 between the first and second samples to obtain a ΔIGFBP-3 value; and
   (d) comparing the ΔIGFBP-3 value from the patient sample against a reference standard of ΔIGFBP-3 from a control population,
wherein the patient is diagnosed as having inducible ischaemia if there is an increase in the ΔIGFBP-3 in the patient sample relative to the reference standard from the control population and wherein, if the patient presented, also an increase in heart rate level between the peak heart rate level determined during the stress test and the resting heart rate level determined after the stress test.

In further aspect of the present invention there is provided a method for diagnosing inducible ischaemia in a patient, the method comprising:
(i) subjecting a patient suspected of having inducible ischaemia to a stress test;
(ii) measuring a peak heart rate level in the patient during the stress test;
(iii) determining if the patient has a transient ischaemia dilation;
(iv) measuring a resting heart rate level in the patient after the stress test;
(v) determining a delta insulin-like growth factor binding protein 3 (ΔIGFBP-3) value which comprises:
   (a) determining the concentration of IGFBP-3 from a first sample obtained from the patient before the stress test;
   (b) determining the concentration of IGFBP-3 from a second sample obtained from the patient after the stress test;
   (c) determining the concentration difference in IGFBP-3 between the first and second samples to obtain a ΔIGFBP-3 value; and
   (d) comparing the ΔIGFBP-3 value from the patient sample against a reference standard of ΔIGFBP-3 from a control population,
wherein, where the patient has confirmed transient ischaemia and an increase in heart rate between peak heart rate (ii) and resting heart rate (iv) together with an increase in the ΔIGFBP-3 in the patient sample relative to the reference standard from the control population (v), is diagnostic that the patient has inducible ischaemia.

In further aspect of the present invention there is provided a method for diagnosing inducible ischaemia in a patient, the method comprising:
(i) measuring a resting heart rate level in a patient suspected of having inducible ischaemia;
(ii) subjecting the patient to a stress test;
(iii) measuring a peak heart rate level in the patient during the stress test;
(iv) determining a delta insulin-like growth factor binding protein 3 (ΔIGFBP-3) value which comprises:
   (a) determining the concentration of IGFBP-3 from a first sample obtained from the patient before the stress test;
   (b) determining the concentration of IGFBP-3 from a second sample obtained from the patient after the stress test;
   (c) determining the concentration difference in IGFBP-3 between the first and second samples to obtain a ΔIGFBP-3 value; and
   (d) comparing the ΔIGFBP-3 value from the patient sample against a reference standard of ΔIGFBP-3 from a control population,
wherein, where the patient has a decreased heart rate level between the resting heart rate level (i) and the peak heart rate level (iii) together with an increase in the ΔIGFBP-3 in the patient sample relative to the reference standard from the control population (iv), is diagnostic that the patient has inducible ischaemia.

In further aspect of the present invention there is provided a method for diagnosing inducible ischaemia in a patient, the method comprising:
(i) measuring a resting heart rate level in a patient suspected of having inducible ischaemia;
(ii) subjecting the patient to a stress test;
(iii) determining if the patient has a transient ischaemia dilation;
(iv) measuring a resting heart rate level in the patient after the stress test;
(v) determining a delta insulin-like growth factor binding protein 3 (ΔIGFBP-3) value which comprises:
   (a) determining the concentration of IGFBP-3 from a first sample obtained from the patient before the stress test;
   (b) determining the concentration of IGFBP-3 from a second sample obtained from the patient after the stress test;
   (c) determining the concentration difference in IGFBP-3 between the first and second samples to obtain a ΔIGFBP-3 value; and
   (d) comparing the ΔIGFBP-3 value from the patient sample against a reference standard of ΔIGFBP-3 from a control population,
wherein, where the patient has a confirmed transient ischaemia dilation and a decreased heart rate level between the resting heart rate level (i) and the peak heart rate level (iv) together with an increase in the ΔIGFBP-3 in the patient sample relative to the reference standard from the control population (v), is diagnostic that the patient has inducible ischaemia.

In a related aspect, the present invention provides a method for diagnosing inducible ischaemia in a patient, typically a patient suspected of having inducible ischaemia, the method comprising:
determining a delta insulin-like growth factor binding protein 3 (ΔIGFBP-3) value which comprises:
   (a) determining the concentration of IGFBP-3 from a first sample obtained from the patient before a stress test;
   (b) determining the concentration of IGFBP-3 from a second sample obtained from the patient after the stress test;
   (c) determining the concentration difference in IGFBP-3 between the first and second samples to obtain a ΔIGFBP-3 value; and
   (d) comparing the ΔIGFBP-3 value from the patient sample against a reference standard of ΔIGFBP-3 from a control population,
wherein the patient is diagnosed as having inducible ischaemia if there is an increase in the ΔIGFBP-3 in the patient sample relative to the reference standard from the control population and wherein, if the patient presented, also an decrease in heart rate level between the resting heart rate level determined before the stress test and the peak heart rate level determined during the stress test.

In another example according to these and other aspects of the present invention, the stress test is induced using an exercise stress test or a pharmacological stress test.

An example of an exercise induced stress test includes most gym equipment including a stationary exercise bicycle, a treadmill, a stepping machine and/or a rowing machine.

An example of a pharmacological stress test includes treatment with adenosine or dobutamine.

In yet a further example according to these and other aspects of the present invention, determination of transient ischaemia dilation is performed using spectral or ultrasound imaging.

The term “index presentation” as used herein is intended to mean the point at which the patient presents with a complaint of chest pain and information about the cardiac status of the patient is measured, including the patient's IGFBP-3 levels. This may be at presentation to (e.g.) an emergency department, a clinic, a hospital, a surgery, a doctor's practice, a doctor or any other relevant medical forum. However, and importantly, in the context of the present invention this term does not preclude repeat testing using the assays described herein where the patient complains of subsequent chest pain during the hospital admission period or during a follow-up consultation.

The term “within about two hours [of index presentation]” as used herein is intended to mean any point on a time continuum measured in seconds, minutes, hours up to and including about ten hours, preferably five hours, more preferably two hours within presentation by the patient to an emergency department, clinic, hospital, surgery, doctor's practice, doctor or any other relevant medical forum or within a similar time frame following a subsequent or isolated complaint of chest pain.

For any avoidance of doubt the term “about” as used in the context of (e.g.) “about two hours” does not mean that the time continuum is limited to two hours, and it may exceed this time limit, for example, by a minute or tens of minutes. For example, “about two hours” could mean 110, 111, 112, 113, 114, 115, 116, 117, 118, 119, 120, 121, 122, 123, 124, 125, 126, 127, 128, 129 or 130 minutes.

In an example according to various aspects of the present invention, the amount, level or concentration of IGFBP-3 is determined using an immunological assay or mass spectrometry.

In another example according to these and other aspects of the present invention, the IGFBP-3 is a peptide defined by SEQ ID NO: 1, and includes fragments thereof. The fragments of IGFBP-3 may be, for example, cleavage fragments including proteolytic fragments of IGFBP-3.

In a further example according to these and other aspects of the present invention described herein, the patient sample is selected from the group consisting of plasma, serum, whole blood, arterial blood, venous blood, saliva, bone marrow tissue, heart tissue, vascular tissue and interstitial fluid sample.

In a related example, the patient sample is a circulating sample selected from plasma, serum, whole blood, arterial blood and venous blood.

In another example according to this aspect of the present invention, the level of IGFBP-3 in the patient sample is at least two, at least three, at least four, at least five, at least six, at least seven, at least eight, at least nine or at least ten times lower than the level of IGFBP-3 in the reference standard of IGFBP-3 from a control population.

In another example according to this aspect of the present invention, the level of IGFBP-3 in the patient sample is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or at least 10 times lower than the level of IGFBP-3 in the reference standard of IGFBP-3 from a control population.

In another example according to this aspect of the present invention, the level of IGFBP-3 in the patient sample is at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 times lower than the level of IGFBP-3 in the reference standard of IGFBP-3 from a control population.

In a further example according to this aspect of the present invention, the ΔIGFBP-3 in the patient sample is at least two, at least three, at least four, at least five, at least six, at least seven, at least eight, at least nine or at least ten times higher than the level of ΔIGFBP-3 in the reference standard obtained from a control population.

In a further example according to this aspect of the present invention, the ΔIGFBP-3 in the patient sample is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or at least 10 times higher than the level of IGFBP-3 in the reference standard of ΔIGFBP-3 from a control population.

In a further example according to this aspect of the present invention, the level of ΔIGFBP-3 in the patient sample is at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 times higher than the level of ΔIGFBP-3 in the reference standard of ΔIGFBP-3 from a control population.

In another example according to this aspect of the present invention, the level of IGFBP-3 in the patient sample is between about 0.01 and 100 ug/mL, and includes without limitation about 0.01, 0.05, 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95 or about 100 ug/mL.

In yet another example according to this aspect of the present invention, the reference standard is from a control population of sex and age-matched subjects who have not been identified as having unstable angina or a history of unstable angina, or a control population of sex and age-matched subjects who are not predisposed to developing cardiac disease including unstable angina, as measured by one or more risk factors.

In a further example according to this aspect of the present invention, the reference standard is the mean IGFBP-3 level or ΔIGFBP-3 from a control population or the median IGFBP-3 or ΔIGFBP-3 level from a control population.

Advantageously, the diagnosis of unstable angina made in accordance with methods described herein may be useful to inform a therapeutic regime to control, reverse, mitigate or treat unstable angina in the patient.

Accordingly, in another aspect of the present invention there is provided a method for diagnosing and treating unstable angina pectoris in a patient, the method comprising:
(i) measuring the level of IGFBP-3 in a patient sample;
(ii) comparing the level of IGFBP-3 in the patient sample against a reference standard of IGFBP-3 from a control population, wherein a decrease in the level of IGFBP-3 in the patient sample measured relative to the reference standard from the control population is diagnostic that the patient has unstable angina pectoris; and
(iii) administering a therapeutic regime to the patient to control, reverse, mitigate or treat the unstable angina pectoris.

In yet another aspect of the present invention there is provided a method for diagnosing and treating unstable angina pectoris in a patient, the method comprising:
(i) measuring a delta-IGFBP-3 (ΔIGFBP-3) value in a patient sample;
(ii) comparing the ΔIGFBP-3 value in the patient sample against a ΔIGFBP-3 value from a control population, wherein an increase in the level of ΔIGFBP-3 in the patient sample measured relative to the reference standard from the control population is diagnostic that the patient has unstable angina pectoris; and
(iii) administering a therapeutic regime to the patient to control, reverse, mitigate or treat the unstable angina pectoris.

In certain examples according to the above aspects of the present invention, the methods further comprise interrogating one or more of a history of angina, an abnormal electrocardiogram, an increased heart rate, dyslipidemia, history of dyslipidemia, abnormal levels of high-density lipoprotein and a diagnosis of ischaemia, optionally by imaging.

The therapeutic regimes administered in accordance with the methods of the present invention include, by way of illustration and example only, those outlined by the Mayo Clinic in its medication guidelines:
https://www.mayoclinic.org/diseases-conditions/myocardial-ischemia.

By way of illustration only, the therapeutic regimes administered in accordance with the present invention include administration of one or more drugs selected from the group consisting of aspirin, nitrates, beta-blockers, calcium channel blockers, cholesterol lowering medications, angiotensin-converting enzyme inhibitors, ranolazine, and any combination thereof.

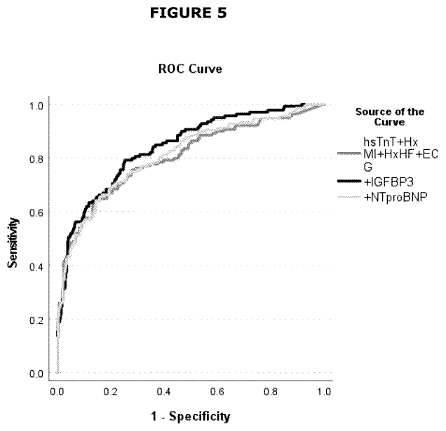

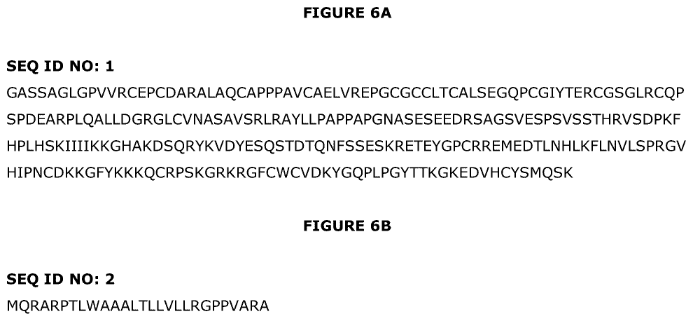

In reference to Example 5, read in conjunction with FIG. 5, the Applicants have also identified a possible utility for IGFBP-3 to improve existing clinical diagnoses of acute coronary syndromes, particularly in an “all-comers” patient cohort who present to (e.g.) to an emergency department, a clinic, a hospital, a surgery, a doctor's practice, a doctor or any other relevant medical forum with a complaint of chest pain, and information about the cardiac status of the patient is measured, including the patient's IGFBP-3 levels, or and a time when a patient presents with a complaint of chest pain during the hospital admission period or during a follow-up consultation and no triage for myocardial infarction has yet been performed.

Accordingly, in yet a further aspect of the present invention there is provided a method for diagnosing an acute coronary syndrome in a patient, the method comprising:
(i) measuring the level of insulin-like growth factor binding protein 3 (IGFBP-3) in a sample obtained from the patient; and
(ii) comparing the level of IGFBP-3 in the patient sample against a reference standard of IGFBP-3 from a control population,
wherein, an increase in the level of IGFBP-3 in the patient sample measured relative to the reference standard from the control population when considered in combination with elevated levels of troponin, an abnormal electrocardiogram, a history of heart failure and/or a history of myocardial infarction, is diagnostic that the patient has an acute coronary syndrome.

In an example according to this and other aspects of the present invention, Troponin includes Troponin T (TnT) and Troponin I (TnI). In a further related example, TnT is high sensitivity Troponin T (hsTnT), and TnI is high sensitivity Troponin I (hsTnI).

### Antibodies and Antigen Binding Fragments

The present invention contemplates various antibodies and antigen-binding fragments thereof which selectively bind to IGFBP-3 or antigenic variants of IGFBP-3 including IGFBP-3 isoforms.

Accordingly, in yet another aspect of the present invention there is provided an antibody or antigen-binding fragment which selectively binds to IGFBP-3.

In yet a further aspect of the present invention there is provided a monoclonal antibody, a polyclonal antibody, a chimeric antibody or a humanized antibody which selectively binds to IGFBP-3, or an antigen-binding fragment of a monoclonal, polyclonal, chimeric or humanized antibody which selectively binds to IGFBP-3.

In yet another aspect of the present invention there is provided a monoclonal antibody or antigen-binding fragment thereof which selectively binds to IGFBP-3.

In yet a further aspect of the present invention there is provided an antibody or antigen-binding fragment which selectively binds to IGFBP-3, which antibody or antigen-binding fragment comprises a detectable label.

In yet another aspect of the present invention there is provided an antibody or antigen-binding fragment which selectively binds to IGFBP-3, which antibody or antigen-binding fragment is immobilized on a solid phase.

In yet a further aspect of the present invention there is provided a binding agent comprising a peptide framework comprising one or more complementarity determining regions derived from an antibody which selectively binds to IGFBP-3.

In yet a further aspect of the present invention there is provided a binding agent comprising a peptide framework comprising at least three complementarity determining regions derived from an antibody which selectively binds to IGFBP-3.

As noted above, antibody or antibodies as used herein refers to a peptide or polypeptide derived from, modelled after or substantially encoded by an immunoglobulin gene or immunoglobulin genes, or fragments thereof, capable of specifically binding an antigen or epitope. As foreshadowed in the definition section of this specification, the term antibody includes antigen binding fragments such as, for example, fragments, subsequences, complementarity determining regions (CDRs) that retain capacity to bind to an antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR). Single chain antibodies are also included by reference in the term “antibody.”

Also included is antiserum obtained by immunizing an animal such as a mouse, rat or rabbit with an antigen, such as for example, IGFBP-3. In brief, methods of preparing polyclonal antibodies are known to a person skilled in the art. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include IGFBP-3, antigenic variants thereof or a fusion protein thereof. It may be useful to conjugate the immunizing agent to a protein known to be immunogenic in the mammal being immunized. Examples of such immunogenic proteins include but are not limited to keyhole limpet hemocyanin, bovine serum albumin, bovine thyroglobulin, and soybean trypsin inhibitor. Examples of adjuvants that may be employed include Freund's complete adjuvant and MPL TDM adjuvant (monophosphoryl Lipid A, synthetic trehalose dicorynomycolate). The immunization protocol may be selected by one skilled in the art without undue experimentation.

Monoclonal antibodies may be prepared using hybridoma methods well known in the art. The hybridoma cells may be cultured in a suitable culture medium, alternatively, the hybridoma cells may be grown in vivo as ascites in a mammal. Preferred immortalized cell lines are murine myeloma lines, which can be obtained, for example, from the American Type Culture Collection, Virginia, USA. Immunoassays may be used to screen for immortalized cell lines that secrete the antibody of interest. Sequences of IGFBP-3 or antigenic variants thereof may be used in screening.

Well known means for establishing binding specificity of monoclonal antibodies produced by the hybridoma cells include immunoprecipitation, radiolinked immunoassay (RIA), enzyme-linked immunoabsorbent assay (ELISA) and Western blot. For example, as noted above, the binding affinity of the monoclonal antibody can, for example, be determined by the Scatchard analysis. Samples from immunised animals may similarly be screened for the presence of polyclonal antibodies.

Monoclonal antibodies can also be obtained from recombinant host cells. DNA encoding the antibody can be obtained from a hybridoma cell line. The DNA is then placed into an expression vector, transfected into host cells (e.g., COS cells, CHO cells, E. coli cells) and the antibody produced in the host cells. The antibody may then be isolated and/or purified using standard techniques.

The monoclonal antibodies or fragments may also be produced by recombinant DNA means. DNA modifications such as substituting the coding sequence for human heavy and light chain constant domains in place of the homologous murine sequences are also possible. The antibodies may be monovalent antibodies. Methods for preparing monovalent antibodies are well known in the art. Production of chimeric, bivalent antibodies and multivalent antibodies are also contemplated herein.

Other known art techniques for monoclonal antibody production such as from phage libraries, may also be used.

The monoclonal antibodies secreted by the cells may be isolated or purified from the culture medium or ascites fluid by conventional immunoglobulin purification procedures such as, for example, reverse phase HPLC, protein A-Sepharose, hydroxyapatite chromatography, gel electrophoresis, dialysis, or affinity chromatography.

Bispecific antibodies may also be useful. These antibodies are monoclonal, preferably human or humanized, antibodies that have binding specificities for at least two different antigens. Antibodies with greater than two specificities for example trispecific antibodies are also contemplated herein.

Antibodies used in the immunoassays described herein selectively bind to IGFBP-3. The term “selectively binds” is not intended to indicate that an antibody binds exclusively to its intended target since, as noted above, an antibody binds to any polypeptide displaying the epitope(s) to which the antibody binds. Rather, an antibody “selectively binds” if its affinity for its intended target is about 5-fold greater when compared to its affinity for a non-target molecule which does not display the appropriate epitope(s). In certain examples, the affinity of the antibody will be at least about 5 fold, preferably 10 fold, more preferably 25-fold, even more preferably 50-fold, and most preferably 100-fold or more, greater for a target molecule than its affinity for a non-target molecule. In other examples, antibodies bind with affinities of at least about 10⁻⁶M, or 10⁻⁷M, or at least about 10⁻⁸M, or 10⁻⁹M, or 10⁻¹⁰ M, or 10⁻¹¹M or 10⁻¹²M.

Affinity is calculated as K_{d}=k_{off}/kₒₙ (k_{off} is the dissociation rate constant, Kₒₙ is the association rate constant and K_{d} is the equilibrium constant). Affinity can be determined at equilibrium by measuring the fraction bound (r) of labelled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art.

Numerous publications discuss the use of phage display technology to produce and screen libraries of polypeptides for binding to a selected analyte. A basic concept of phage display methods is the establishment of a physical association between DNA encoding a polypeptide to be screened and the polypeptide. This physical association is provided by the phage particle, which displays a polypeptide as part of a capsid enclosing the phage genome that encodes the polypeptide. The establishment of a physical association between polypeptides and their genetic material allows simultaneous mass screening of very large numbers of phage bearing different polypeptides. Phage displaying a polypeptide with affinity to a target binds to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labelled secondary antibody (for example, an anti-mouse antibody conjugated to alkaline phosphatase if the raised antibodies are mouse antibodies) is added to the wells and incubated for about 30 min and then washed. Substrate is added to the wells and a colour reaction will appear where antibody to the immobilized polypeptide(s) is present.

The antibodies so identified may then be further analysed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interact with one another sterically, etc., assay performance of an antibody may be a more important measure than absolute affinity and specificity of an antibody.

### Aptamers

The present invention also contemplates aptamers that selectively bind to IGFBP-3 or antigenic variants of IGFBP-3 including IGFBP-3 isoforms.

In yet another aspect of the present invention there is provided an aptamer or aptamer ligand binding domain which selectively binds to IGFBP-3.

Nucleic acid aptamers are nucleic acid molecules that have been engineered through repeated rounds of in vitro selection, SELEX (systematic evolution of ligands by exponential enrichment) to bind to various molecular targets such as small molecules, proteins, nucleic acids, and even cells, tissues and organisms. Aptamers offer molecular binding and recognition equivalent to antibodies. In addition to their discriminate recognition, aptamers offer advantages over antibodies as they can be engineered completely in vitro, are readily produced by chemical synthesis, possess desirable storage properties, and elicit little or no immunogenicity in therapeutic applications.

According to an example of the present invention, the aptamer is a monomer (one unit). According to another example of the invention, the aptamer is a multimeric aptamer. The multimeric aptamer may comprise a plurality of aptamer units (mers). Each of the plurality of units of the aptamer may be identical. In such a case the multimeric aptamer is a homomultimer having a single specificity but enhanced avidity (multivalent aptamer).

Alternatively, the multimeric aptamer may comprise two or more aptameric monomers, wherein at least two mers of the multimeric aptamer are non-identical in structure, nucleic acid sequence or both. Such a multimeric aptamer is referred to herein as a heteromultimer. The heteromultimer may be directed to a single binding site i.e., monospecific (such as to avoid steric hindrance). The heteromultimer may be directed to a plurality of binding sites i.e., multispecific. The heteromultimer may be directed to a plurality of binding sites on different analytes. Further description of a multimeric aptamer is provided below.

A plurality of multimeric aptamers may be conjugated to form a conjugate of multimeric aptamers. The multimeric aptamer may comprise, two (dimer), three (trimer), four (tetramer), five (pentamer), six (hexamer), and even more units.

Aptamers of the invention can be synthesized and screened by any suitable methods in the art.

For example, aptamers can be screened and identified from a random aptamer library by SELEX (systematic evolution of ligands by exponential enrichment). Aptamers that bind to an antigen of interest can be suitably screened and selected by a modified selection method herein referred to as cell-SELEX or cellular-SELEX.

A random aptamer library can be created that contains monomeric, dimeric, trimeric, tetrameric or other higher multimeric aptamers. A random aptamer library (either ssDNA or RNA) can be modified by including oligonucleotide linkers to link individual aptamer monomers to form multimeric aptamer fusion molecules. In other examples, a random oligonucleotide library is synthesized with randomized 45 nt sequences flanked by defined 20 nt sequences both upstream and downstream of the random sequence, i.e., known as 5′-arm and 3′-arm, which are used for the amplification of selected aptamers. A linking oligonucleotide (i.e., linker) is designed to contain sequences complementary to both 5′-arm and 3′-arm regions of random aptamers to form dimeric aptamers. For trimeric or tetrameric aptamers, a small trimeric or tetrameric (i.e., a Holiday junction-like) DNA nanostructure is engineered to include sequences complementary to the 3′-arm region of the random aptamers, therefore creating multimeric aptamer fusion through hybridization. In addition, 3-5 or 5-10 dT rich nucleotides can be engineered into the linker polynucleotides as a single stranded region between the aptamer-binding motifs, which offers flexibility and freedom of multiple aptamers to coordinate and synergize multivalent interactions with cellular ligands or receptors. Alternatively, multimeric aptamers can also be formed by mixing biotinylated aptamers with streptavidin.

A modified cellular SELEX procedure can be employed to select target-binding aptamers. Multimeric aptamers may be multivalent but be of single binding specificity (i.e., homomultimeric aptamers). Alternatively, the multimeric aptamer may be multivalent and multi-specific (i.e., heteromultimeric aptamers).

Thus, each monomer of the homomultimeric aptamer binds the target protein (e.g., IGFBP-3 as well as antigenic variants thereof) in an identical manner. Thus according to an example of the invention, all monomeric components of the homomultimeric aptamer are identical.

Conversely, a heteromultimeric aptamer comprises a plurality of monomeric aptamers at least two of which bind different sites on a single target protein or bind at least two different target proteins.

Selection of RNA-aptamers is well-established using protocols described in the scientific literature.

In certain examples, a suitable nucleotide length for an aptamer ranges from about 15 to about 100 nucleotide (nt), and in various other examples, 12-30, 14-30, 15-30 nt, 30-100 nt, 30-60 nt, 25-70 nt, 25-60 nt, 40-60 nt, or 40-70 nt in length.

Multimerization can be done at the library level as follows.

In certain examples, a linker polynucleotide has a length between about 5 nucleotides (nt) and about 100 nt; in various examples, 10-30 nt, 20-30 nt, 25-35 nt, 30-50 nt, 40-50 nt, 50-60 nt, 55-65 nt, 50-80 nt, or 80-100 nt. It is within the ability of one of skill in the art to adjust the length of the linker polynucleotide to accommodate each monomeric aptamer in the multimeric structure.

In certain examples, the multimeric aptamers can be identified and screened from a random multimeric aptamer library as described herein. In other examples, the monomeric aptamers are linked to each other by one or a plurality of linker polynucleotides to form multimeric aptamers. Monomeric aptamers can be linked to form multimeric aptamers by any suitable means and in any configurations.

It will be appreciated that the monomeric structures of the invention can be further multimerized by post SELEX procedures.

Multimers can be linearly linked by continuous linear synthesis of DNA without spacers or with nucleic acid spacers. Aptamer synthesis usually relies on standard solid phase phosphoramitide chemistry.

Thus, dimers, trimers and tetramers or higher oligomeric structures (e.g., pentamers, hexamers, heptamers, octamers etc.) can be linked by a polymeric spacer.

In certain examples, the aptamers are further modified to protect the aptamers from nuclease and other enzymatic activities. The aptamer sequence can be modified by any suitable methods known in the art. For example, phosphorothioate can be incorporated into the backbone, and 5′-modified pyrimidine can be included in 5′ end of ssDNA for DNA aptamer. For RNA aptamers, modified nucleotides such as substitutions of the 2′-OH groups of the ribose backbone, e.g., with 2′-deoxy-NTP or -fluoro-NTP, can be incorporated into the RNA molecule using T7 RNA polymerase mutants. The resistance of these modified aptamers to nuclease can be tested by incubating them with either purified nucleases or nuclease from mouse serum, and the integrity of aptamers can be analyzed by gel electrophoresis.

The monomeric or multimeric aptamer of the invention can be further attached or conjugated to a detectable or therapeutic moiety (i.e., a pharmaceutical moiety).

Thus, as noted above, a diagnostic or therapeutic moiety can be attached to an aptamer embodied herein to provide additional biological activity, such as for diagnosing, preventing, or treating a condition or disease. In one example a diagnostic moiety such as a detectable moiety e.g., label (e.g., His tag, flag tag), fluorescent, radioactive, biotin/avidin etc., can be bound to the aptamer, and imaging, immunohistochemistry, or other invasive or non-invasive methods used to identify the location(s) and extend of binding of the conjugate to locations within the body. For therapeutic uses, a cytotoxic agent such as a chemotherapeutic agent, radioactive moiety, toxin, antibody, nucleic acid silencing agents e.g., small interfering RNA (siRNA) or other molecule with therapeutic activity when delivered to cells expressing a molecule to which the aptamer is targeted, may be used to enhance the therapeutic activity of the aptamer or provide a biological activity where the aptamer is providing the targeting activity. Moreover, other conjugates to the aptamers described herein are contemplated, such as but not limited to scaffolds, sugars, proteins, antibodies, polymers, and nanoparticles, each of which have art-recognized therapeutic or diagnostic utilities and can be targeted to particular sites in vivo using an aptamer embodied herein.

### Detection of Binding Agents Including Peptide Binding Assays

The present invention includes use of a detection system involving the binding of IGFBP-3 to a binding agent and then detecting the amount of bound peptide. A similar solution is to detect the amount of unbound binding agent in a sample to get an indication of unbound or bound IGFBP-3. It is intended that such alternative methods fall within the scope of the present invention as functional alternatives to directly detecting the amount of bound binding agent. Persons skilled in the art will appreciate that the concentration of IGFBP-3 in a sample can be readily calculated from the amount of IGFBP-3 in a sample when the sample volume is known.

In the assays, methods and kits according to the present invention, the measuring steps comprise detecting binding between IGFBP-3 and a binding agent that binds, selectively or specifically, to IGFBP-3, and has low cross-reactivity with other markers of biological events.

Accordingly, in another aspect of the present invention there is provided an assay method for measuring the level of IGFBP-3 in a sample, the method comprising:
(i) contacting a sample with a binding agent which selectively binds to IGFBP-3; and
(ii) determining the amount of binding agent bound to IGFBP-3 so as to establish the level of IGFBP-3 in the sample.

In certain examples, the binding agent is an antibody or an antigen-binding fragment thereof. The antibody may be a monoclonal, polyclonal, chimeric or humanized antibody or antigen-binding fragment thereof. As such, in one example the assay, as well as methods involving assays, of the present invention is an immunoassay.

As such, in a further aspect of the present invention there is provided an assay method for measuring the level of IGFBP-3 in a sample, the method comprising:
(i) contacting a sample with an antibody or aptamer which selectively binds to IGFBP-3; and
(ii) determining the amount of antibody or aptamer bound to IGFBP-3 so as to establish the level of IGFBP-3 in the sample.

The antibodies of the present invention are particularly useful in immunoassays for determining the presence and/or amount of IGFBP-3 in a sample. Due to variable binding affinities of different antibodies, the person skilled in the art will appreciate that a standard binding curve of measured values versus amount of IGFBP-3 in a sample should be established for a particular antibody to enable the amount of IGFBP-3 in a sample to be determined. Such a curve is used to determine the true amount of IGFBP-3 in a sample.

Sample materials include biological fluids but are not limited thereto. In terms of the present invention, usually a biological fluids are selected from whole blood, plasma or serum.

Immunoassays specific for IGFBP-3 usually will require the production of antibodies that specifically bind to IGFBP-3. The antibodies can be used to construct immunoassays with broad specificity, as in competitive binding assays below, or used in conjunction with other antibodies described below in sandwich type assays to produce assays specific to IGFBP-3. The person skilled in the art will appreciate that non-competitive assays are also possible. The latter antibodies for sandwich immunoassays include those specific for amino acid sequences including SEQ ID NO:1.

In another example, indicators may also be used. Indicators may be employed in ELISA and RIA assay formats.

Polyclonal and monoclonal antibodies can be used in competitive binding or sandwich type assays. In one example of this method a liquid sample is contacted with the antibody and simultaneously or sequentially contacted with a labelled IGFBP-3 or modified peptide containing the epitope recognised by the antibody.

The label can be a radioactive component such as ¹²⁵I, ¹³¹I, ³H, ¹⁴C or a non-radioactive component that can be measured by time resolved fluorescence, fluorescence, fluorescence polarisation, luminescence, chemiluminescence or colorimetric methods. These compounds include europium or other actinide elements, acrinidium esters, fluorescein, or radioactive material such as those above, that can be directly measured by radioactive counting, measuring luminescent or fluorescent light output, light absorbance etc. The label can also be any component that can be indirectly measured such as biotin, digoxin, or enzymes such as horseradish peroxidase, alkaline phosphatase. These labels can be indirectly measured in a multitude of ways. Horseradish peroxidase for example can be incubated with substrates such as o-Phenylenediamine Dihyhdrochloride (OPD) and peroxide to generate a coloured product whose absorbance can be measured, or with luminol and peroxide to give chemiluminescent light which can be measured in a luminometer. Biotin or digoxin can be reacted with binding agents that bind strongly to them; e.g. avidin will bind strongly to biotin. These binding agents can in turn be covalently bound or linked to measurable labels such as horseradish peroxidase or other directly or indirectly measured labels as above. These labels and those above may be attached to the peptide or protein: during synthesis, by direct reaction with the label, or through the use of commonly available crosslinking agents such as MCS and carbodiimide, or by addition of chelating agents.

Following contact with the antibody, usually for 18 to 25 hours at 4° C., or 1 to 240 minutes at 30° C. to 40° C., the labelled peptide bound to the binding agent (antibody) is separated from the unbound labelled peptide. In solution phase assays, the separation may be accomplished by addition of an anti-gamma globulin antibody (second-antibody) coupled to solid phase particles such as cellulose, or magnetic material. The second-antibody is raised in a different species to that used for the primary antibody and binds the primary antibody. All primary antibodies are therefore bound to the solid phase via the second antibody. This complex is removed from solution by centrifugation or magnetic attraction and the bound labelled peptide measured using the label bound to it. Other options for separating bound from free label include formation of immune complexes, which precipitate from solution, precipitation of the antibodies by polyethyleneglycol or binding free labelled peptide to charcoal and removal from solution by centrifugation of filtration. The label in the separated bound or free phase is measured by an appropriate method such as those presented above.

Competitive binding assays can also be configured as solid phase assays that are easier to perform and are therefore preferable to those above. This type of assay use a solid support including plates with wells (commonly known as ELISA or immunoassay plates), solid beads or the surfaces of tubes. The primary antibody is either adsorbed or covalently bound to the surface of the plate, bead or tube, or is bound indirectly through a second anti gamma globulin or anti Fc region antibody adsorbed or covalently bound to the plate. Sample and labelled peptide (as above) are added to the plate either together or sequentially and incubated under conditions allowing competition for antibody binding between IGFBP-3 in the sample and the labelled peptide. Unbound labelled peptide can subsequently be aspirated off and the plate rinsed leaving the antibody bound labelled peptide attached to the plate. The labelled peptide can then be measured using techniques described above.

Sandwich type assays are more preferred for reasons of specificity, speed and greater measuring range. In this type of assay an excess of the primary antibody to IGFBP-3 is attached to the well of an ELISA plate, bead or tube via adsorption, covalent coupling, or an anti Fc or gamma globulin antibody, as described above for solid phase competition binding assays. Sample fluid or extract is contacted with the antibody attached to the solid phase. Because the antibody is in excess this binding reaction is usually rapid. A second antibody to a IGFBP-3 peptide is also incubated with the sample either simultaneously or sequentially with the primary antibody. This second antibody is chosen to bind to a site on IGFBP-3 that is different from the binding site of the primary antibody. These two antibody reactions result in a sandwich with the IGFBP-3 from the sample sandwiched between the two antibodies. The second antibody is usually labelled with a readily measurable compound as detailed above for competitive binding assays. Alternatively a labelled third antibody which binds specifically to the second antibody may be contacted with the sample. After washing the unbound material the bound labelled antibody can be measured by methods outlined for competitive binding assays. After washing away the unbound labelled antibody, the bound label can be quantified as outlined for competitive binding assays.

A dipstick type assay may also be used. These assays are well known in the art. They may for example, employ small particles such as gold or coloured latex particles with specific antibodies attached. The liquid sample to be measured may be added to one end of a membrane or paper strip preloaded with the particles and allowed to migrate along the strip. Binding of the antigen in the sample to the particles modifies the ability of the particles to bind to trapping sites, which contain binding agents for the particles such as antigens or antibodies, further along the strip. Accumulation of the coloured particles at these sites results in colour development are dependent on the concentration of competing antigen in the sample. Other dipstick methods may employ antibodies covalently bound to paper or membrane strips to trap antigen in the sample. Subsequent reactions employing second antibodies coupled to enzymes such as horse radish peroxidase and incubation with substrates to produce colour, fluorescent or chemiluminescent light output will enable quantitation of antigen in the sample.

The present invention further contemplates a peptide complex comprising IGFBP-3 bound to a binding agent which selectively binds to IGFBP-3.

Accordingly, in a further aspect of the present invention there is provided a peptide complex comprising IGFBP-3 bound to a binding agent which selectively binds to IGFBP-3.

In yet a further aspect of the present invention there is provided a peptide complex comprising IGFBP-3 bound to an antibody or antigen binding fragment thereof which selectively binds to IGFBP-3.

In another further aspect of the present invention there is provided a peptide complex comprising IGFBP-3 bound to a polyclonal antibody or antigen-binding fragment thereof which selectively binds to IGFBP-3.

In another further aspect of the present invention there is provided a peptide complex comprising IGFBP-3 bound to a monoclonal antibody or antigen-binding fragment thereof which selectively binds to IGFBP-3.

In yet another aspect of the present invention there is provided a peptide complex comprising IGFBP-3 bound to an aptamer which selectively binds to IGFBP-3.

Further contemplated by the present invention is methods for detection of a peptide complex comprising IGFBP-3 bound to a binding agent which selectively binds to IGFBP-3.

Accordingly, in a further aspect of the present invention there is provided a method for detecting a peptide complex from a biological sample, wherein the peptide complex comprises IGFBP-3 bound to a binding agent which selectively binds to IGFBP-3.

In yet a further aspect of the present invention there is provided a method for detecting a peptide complex from a biological sample, wherein the peptide complex comprises IGFBP-3 bound to an antibody or antigen-binding fragment thereof which selectively binds to IGFBP-3.

In yet a further aspect of the present invention there is provided a method for detecting a peptide complex from a biological sample, wherein the peptide complex comprises IGFBP-3 bound to a monoclonal antibody or antigen-binding fragment thereof which selectively binds to IGFBP-3.

In yet a further aspect of the present invention there is provided a method for detecting a peptide complex from a biological sample, wherein the peptide complex comprises IGFBP-3 bound to a polyclonal antibody or antigen-binding fragment thereof which selectively binds to IGFBP-3.

In yet a further aspect of the present invention there is provided a method for detecting a peptide complex from a biological sample, wherein the peptide complex comprises IGFBP-3 bound to an aptamer which selectively binds to IGFBP-3.

According to these and other aspects, the present invention further contemplates the methods described herein in which one or more risk factors are also interrogated. This includes, without limitation, one or more clinical risk factors selected from a history of angina, a decreased heart rate relative to a reference standard, a decreased level of high-density lipoprotein relative to a reference standard, an abnormal electrocardiogram, a diagnosis of ischaemia, optionally by imaging, and a history of dyslipidemia

### Detection of IGFBP-3 by Mass Spectrometry

Given the relative abundance of IGFBP-3 in circulation (i.e. 1-1000 uM range), and in particular the amount/concentration of this peptide fragment detected in plasma levels, measurement of IGFBP-3 in a patient sample is particularly suited to detection using non binding-agent assays including, without limitation, mass spectrometry, x-ray diffraction nuclear magnetic resonance and high performance liquid chromatography.

The precise molecular mass of IGFBP-3 has been determined as ˜40-43 kDa, and accordingly it is possible to detect the presence of this fragment in a biological sample of interest using mass spectrometry including tandem mass spectrometry or “MS/MS”. MS/MS is a technique in instrumental analysis where two or more mass analyzers are coupled together using an additional reaction step to increase their abilities to analyse chemical samples. The molecules of a given sample are ionized and the first spectrometer (designated MS1) separates these ions by their mass-to-charge ratio (often given as m/z or m/Q). Ions of a particular m/z-ratio coming from MS1 are selected and then made to split into smaller fragment ions, e.g. by collision-induced dissociation, ion-molecule reaction, or photodissociation. These fragments are then introduced into the second mass spectrometer (MS2), which in turn separates the fragments by their m/z-ratio and detects them. The fragmentation step makes it possible to identify and separate ions that have very similar m/z-ratios in regular mass spectrometers.

Accordingly, in another aspect of the present invention there is provided a method for diagnosing an acute coronary syndrome in a patient, the method comprising:
(i) measuring the level of IGFBP-3 in a patient sample using mass spectrometry; and
(ii) comparing the level of IGFBP-3 in the patient sample against reference standard of IGFBP-3 from a control population,
wherein, an increase in the level of IGFBP-3 in the patient sample measured relative to the reference standard from the control population reflects that the patient has an acute coronary syndrome.

In yet another aspect of the present invention there is provided a method for diagnosing unstable angina in a patient, the method comprising:
(i) measuring the level of IGFBP-3 in a patient sample using mass spectrometry; and
(ii) comparing the level of IGFBP-3 in the patient sample against reference standard of IGFBP-3 from a control population,
wherein, an increase in the level of IGFBP-3 in the patient sample measured relative to the reference standard from the control population reflects that the patient has an acute coronary syndrome.

In reading this specification in its entirety, it would be appreciated by the skilled person that any method or assay described and claimed herein may be modified to include the step of measuring the level of IGFBP-3 in a patient sample using mass spectrometry, an in particular tandem mass spectrometry or MS/MS. The above statements of invention in relation to detection of IGFBP-3 in a patient sample using mass spectrometry for diagnosing an acute coronary syndrome or for diagnosing unstable angina are merely representative aspects of the present invention.

### Receiver Operating Characteristic (ROC) Analysis

The clinical performance of a laboratory test depends on its diagnostic/prognostic accuracy, or the ability to correctly classify subjects into clinically relevant subgroups. Prognostic accuracy measures the test's ability to correctly distinguish two different conditions of the subjects investigated. Such conditions are for example health and disease or benign versus malignant disease.

In each case, a receiver operating characteristic (ROC) plot depicts the overlap between the two distributions by plotting the sensitivity versus 1-specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1-specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test.

One convenient objective to quantify the diagnostic accuracy of a laboratory test is to express its performance by a single number. The most common global measure is the area under the ROC plot. By convention, this area is always ≥0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

### Test Kits & Articles of Manufacture

Typically, test kits or articles of manufacture will be formatted for assays known in the art, and in certain examples for RIA or ELISA assays, as are known in the art.

Binding agents that selectively bind IGFBP-3 or antigenic variants of IGFBP-3 thereof are desirably included in the test kits or articles of manufacture.

Accordingly, in an aspect of the present invention there is provided a test kit or article of manufacture for diagnosing a cardiac disease in a patient, the test kit or article of manufacture comprising a binding agent which selectively binds to IGFBP-3, and optionally, instructions for how to predict or diagnose the cardiac disease in the patient.

In another aspect of the present invention there is provided a test kit or article of manufacture for diagnosing unstable angina in a patient, the test kit or article of manufacture comprising a binding agent which selectively binds to IGFBP-3, and optionally, instructions for how to diagnose unstable angina in the patient.

In certain aspects, the binding agent is an aptamer or an antibody or antigen-binding fragment which selectively binds to IGFBP-3. The antibody(ies) used in the assays and kits may be monoclonal or polyclonal, for example, and may be prepared in any mammal as described above, and includes antigen binding fragments and antibodies prepared using native and fusion peptides, for example.

Accordingly, in yet another aspect of the present invention there is provided a test kit or article of manufacture for diagnosing a cardiac disease in a patient, the test kit or article of manufacture comprising an antibody or aptamer which selectively binds to IGFBP-3, and optionally, instructions for how to predict or diagnose the cardiac in the patient.

In yet a further aspect of the present invention there is provided a test kit or article of manufacture for diagnosing a cardiac disease in a patient, the test kit or article of manufacture comprising a monoclonal antibody or antigen-binding fragment thereof which selectively binds to IGFBP-3, and optionally, instructions for how to predict or diagnose the cardiac disease in the patient.

In yet a further aspect of the present invention there is provided a test kit or article of manufacture for diagnosing unstable angina in a patient, the test kit or article of manufacture comprising a monoclonal antibody antigen-binding fragment thereof which selectively binds to IGFBP-3, and optionally, instructions for how to diagnose unstable angina in the patient.

The test kits or articles of manufacture may be comprised of one or more containers and may also include collection equipment, for example, bottles, bags (such as intravenous fluids bags), vials, syringes, and test tubes. At least one container will be included and will hold a product which is effective for use in the assays and methods described herein. The product is typically a peptide binding agent, particularly an antibody or antigen-binding fragment of the invention, or a composition comprising any of these. In one example, an instruction or label on or associated with the container indicates that the composition is used for predicting, diagnosing, or monitoring a cardiac disease in the subject. Other components may include needles, diluents and buffers.

The test kits or articles of manufacture may also include detection or measurement means involving one or more additional markers or risk factors for a cardiac disease of interest (e.g.) including heart rate, haemoglobin concentration, blood pressure, age, sex, weight, level of physical activity, family history of events including obesity, diabetes and cardiac events, and levels of circulating Troponin T, Troponin I, NT-proBNP, BNP, BNPsp and BNPsp fragments including BNPsp(17-26). Again, this may include binding agent(s), aptamer(s), antibody(s) and antigen-binding fragment(s) thereof which selectively bind to other biomarker(s) of interest.

In other aspects, the test kits or articles of manufacture according to the present invention comprise a binding agent which selectively binds to IGFBP-3, and optionally instructions for how to:
(i) diagnose an acute coronary syndrome together with other risk factors including, but not limited to, levels of hsTnI, abnormal ECG, history of MI and history of HF;
(ii) diagnose unstable angina pectoris based on a delta-IGFBP-3 value; and
(iii) diagnose unstable angina pectoris together with other risk factors including, but not limited to, a history of angina, abnormal ECG, an increased heart rate, dyslipidemia, a history of dyslipidemia and abnormal levels of high-density lipoprotein.

In certain examples of the test kits or articles of manufacture according to the present invention, the IGFBP-3 binding agent is immobilized on a solid matrix, for example, a porous strip or chip to form at least one detection site for a IGFBP-3 or an antigenic fragment(s) thereof. The measurement or detection region of the porous strip may include a plurality of detection sites, such detection sites containing a detection reagent. The sites may be arranged in a bar, cross or dot or other arrangement. A test strip or chip may also contain sites for negative and/or positive controls. Alternatively, the control sites may be on a different strip or chip. The different detection sites may contain different amounts of immobilized nucleic acids or antibodies, e.g., a higher amount in the first detection site and lower amounts in subsequent sites. Upon the addition of a test sample the number of sites displaying a detectable signal provides a quantitative indication of the amount of a IGFBP-3 or antigenic variant(s) thereof present in the sample.

Also included in the kits or articles of manufacture may be a device for sample analysis comprising a disposable testing cartridge with appropriate components (markers, antibodies and reagents) to carry out sample testing. The device will conveniently include a testing zone and test result window. Immunochromatographic cartridges are examples of such devices. See for example U.S. Pat. Nos. 6,399,398; 6,235,241 and 5,504,013.

Alternatively, the device may be an electronic device which allows input, storage and evaluation of levels of the measured marker against control levels and other marker levels. US 2006/0234315 provides examples of such devices. Also useful in the invention are Ciphergen's Protein Chip® which can be used to process SELDI results using Ciphergen's Protein Chip® software package.

The invention is further described with reference to the following examples. It will be appreciated that the invention as claimed is not intended to be limited in any way by these examples.

### EXAMPLES

### Example 1: Methods and Materials

Levels of IGFBP-3 were measured using RnD Systems Quantikine ELISA (Catalogue #DGB300).

LoD 0.5 ng/mL, ED₅₀ 25 ng/mL, 6 uL sample requirement diluted 100× for assay. 4 hr turnaround, working range 0.8-50 ng/mL. No effect of EDTA, LithHep or serum collection. No cross reactivities with other related proteins, lipids, IgG/M, bilirubin, albumin or drugs such as clopidogrel, aspirin, beta-blockers, ACE inhibitors. Has not been tested for x-reactivity with EPA or Entresto.

| | | | | Intermediate | | |
|---|---|---|---|---|---|---|
| | Repeatability | | | precision | | |
| | (Intra-Assay CV) | | | (Inter-Assay CV) | | |
| Sample | 1 | 2 | 3 | 1 | 2 | 3 |
| n | 20 | 20 | 20 | 40 | 40 | 40 |
| Mean | 4.13 | 12.7 | 31.1 | 4.06 | 11.8 | 28.5 |
| Std Dev | 0.2 | 0.64 | 0.71 | 0.22 | 0.76 | 2.27 |
| CV % | 4.8 | 5 | 2.3 | 5.4 | 6.4 | 8 |

### Example 2: Biomarker Analysis for DX of Non/Cardiac Conditions

IGFBP-3, NT-proBNP and hsTnT were analysed using Receiver Operating Curve (ROC) analysis for their ability to diagnose myocardial infarction (MI), unstable angina pectoris (UAP) and non-cardiac chest pain (NCCP).

Briefly, these experiments involved a patient cohort (“SPACE”) made up of individuals who had presented to an emergency department with a complaint of chest pain. The total number of patients in the SPACE cohort is n=1018, with clinically adjudicated unstable angina pectoris (n=72); myocardial infarction (n=201); non-cardiac chest pain (n=420); undifferentiated chest pain (n=275); and other cardiac conditions (n=50).

The results are presented in FIGS. 2A to 2C, read in conjunction with Tables 1-3, below.

The data reveal that IGFBP-3 had no ability to diagnose myocardial infarction (AUC=0.482±0.023) relative to the “gold standard” high sensitivity Troponin T (AUC=0.931±0.010), and performed marginally worse that NT-proBNP (AUC=0.647±0.022). Refer to Table 1 and FIG. 2A.

TABLE 1
ROC Performance of IGFBP-3 relative
to NT-proBNP and hsTnT for Dx MI

| | | Std. | Asympt. | Asympt. 95% Conf. Int. | |
|---|---|---|---|---|---|
| Variables | AUC | Error | Sig | Lower Bound | Upper Bound |
| IGFBP-3 | 0.482 | 0.023 | 0.444 | 0.437 | 0.528 |
| NT-proBNP | 0.647 | 0.022 | 0.000 | 0.605 | 0.690 |
| hsTnT | 0.931 | 0.010 | 0.000 | 0.912 | 0.951 |

NT-proBNP and hsTnT AUC values were not as powerful as IGFBP-3, which provided superior differentiation of UAP compared with either of NT-proBNP and hsTnT (IGFBP-3 AUC=0.367 which was further removed from the reference line of 0.5).

TABLE 2
ROC Performance of IGFBP-3 relative to
NT-proBNP and hsTnT for Dx UAP

| | | Std. | Asympt. | Asympt. 95% Conf. Int. | |
|---|---|---|---|---|---|
| Variables | AUC | Error | Sig | Lower Bound | Upper Bound |
| IGFBP-3 | 0.367 | 0.035 | 0.000 | 0.300 | 0.435 |
| NT-proBNP | 0.581 | 0.033 | 0.014 | 0.517 | 0.645 |
| hsTnT | 0.407 | 0.031 | 0.003 | 0.346 | 0.468 |

Finally, none of the markers interrogated in these experiments showed any significance for the diagnosis of non-cardiac chest pain with IGFBP-3 (AUC=0.581±0.018), NT-proBNP (AUC=0.262±0.016) and hsTnT (AUC=0.272±0.016). Again, these values are well below what would be considered clinically useful. Refer to Table 3 and FIG. 2C. These data are not surprising given hsTnT and NT-proBNP are “classical” cardiac biomarkers whose AUC performance is driven largely by acute MI, not UAP.

TABLE 3
ROC Performance of IGFBP-3 relative to
NT-proBNP and hsTnT for Dx NCCP

| | | Std. | Asympt. | Asympt. 95% Conf. Int. | |
|---|---|---|---|---|---|
| Variables | AUC | Error | Sig | Lower Bound | Upper Bound |
| IGFBP-3 | 0.581 | 0.018 | 0.000 | 0.545 | 0.617 |
| NT-proBNP | 0.262 | 0.016 | 0.000 | 0.231 | 0.294 |
| hsTnT | 0.272 | 0.016 | 0.000 | 0.245 | 0.304 |

### Example 3: IGFBP-3 ADDS to DX Power of Existing UAP Predictors in “Space” Patient Cohort

For the rapid diagnosis of unstable angina pectoris in SPACE cohort, Applicants looked for clinical diagnostic predictors, independent of the stress testing and imaging results.

Regression analysis revealed Hx of angina (present in 81% of adjudicated cases), abnormal ECG (present in 30% of cases) and history of dyslipidemia (88% of UAP cases) are the significant predictive factors. Refer to Table 4, below.

TABLE 4
Regression Analysis of Risk Factors for Dx of UAP

| Step | | Variables in the Equation | | | | | |
|---|---|---|---|---|---|---|---|
| 1^{a} | | B | S.E. | Wald | df | Sig. | Exp(B) |
| | HxAng | 1.350 | 0.367 | 13.520 | 1 | 0.000 | 3.856 |
| | HxDyslip | 1.093 | 0.433 | 6.387 | 1 | 0.011 | 2.984 |
| | ECG_Change | 1.560 | 0.310 | 25.330 | 1 | 0.000 | 4.760 |
| | GenderBinary | −0.074 | 0.304 | 0.059 | 1 | 0.809 | 0.929 |
| | Calc_Age@Adm | −0.012 | 0.013 | 0.905 | 1 | 0.342 | 0.988 |
| | HxMI | −0.034 | 0.293 | 0.013 | 1 | 0.908 | 0.967 |
| | eGFR_CKDepi | −0.003 | 0.009 | 0.094 | 1 | 0.759 | 0.997 |
| | LogHR | −1.154 | 0.598 | 3.720 | 1 | 0.054 | 0.316 |
| | Constant | 1.546 | 2.966 | 0.272 | 1 | 0.602 | 4.691 |

^{a}variables entered on Step 1: ECG_Change, AngHx, HxDyslip, IGFBP-3_0_Log10, HDL_Log10, Log10_NTproBNP

Using these data, Applicants then built a model (index) to diagnose UAP at index presentation (i.e. first blood draw results). These data are presented in Table 5 and demonstrate that when IGFBP-3 and history of dyslipidemia (±HDL-C) were used to augment history of angina and abnormal ECG, a powerful clinical diagnostic tool was created.

TABLE 5
Model Index to Predict ED Likelihood of UAP on First Blood Draw Results

| | Variables in the Equation | | | | | | | | |
|---|---|---|---|---|---|---|---|---|---|
| | | | | | | | | 95% CI | |
| Step | | | | | | | | Exp(B) | |
| 1^{a} | | B | S.E. | Wald | df | Sig. | Exp(B) | Low. | Upp. |
| | ECG_Change | 1.558 | 0.344 | 20.556 | 1 | 0.000 | 4.747 | 2.421 | 9.309 |
| | HxAng | 1.128 | 0.358 | 9.906 | 1 | 0.002 | 3.090 | 1.531 | 6.240 |
| | HxDyslip | 1.064 | 0.436 | 5.966 | 1 | 0.015 | 2.899 | 1.234 | 6.811 |
| | IGFBP_0_Log10 | −2.128 | 0.567 | 14.092 | 1 | 0.000 | 0.119 | 0.039 | 0.362 |
| | HDL_Log10 | −2.765 | 1.141 | 5.875 | 1 | 0.015 | 0.063 | 0.007 | 0.589 |
| | Log10_NTproBNP | 0.165 | 0.251 | 0.433 | 1 | 0.510 | 1.180 | 0.721 | 1.929 |
| | Log10_hsTnT | 0.080 | 0.356 | 0.051 | 1 | 0.822 | 1.083 | 0.540 | 2.175 |
| | Constant | 2.291 | 1.780 | 1.657 | 1 | 0.198 | 9.887 | | |

Further ROC analyses revealed that the addition of IGFBP-3 to AngHx+ECG improved the diagnostic power of this combination for the diagnosis of UAP, where the AUC increased from AUC=0.728±0.027 to AUC=0.767±0.027. The addition of HxDyslipid and HDL further improved the diagnostic ability with AUC=0.784±0.024 and 0.797±0.024, respectively. Refer to Table 6, below read in conjunction with ROC curve presented in FIG. 3.

TABLE 6
ROC Curve Classification in ED @ T = 0: IGFBP-3 + Current UAP Dx

| | | | | Asympt. 95% | |
|---|---|---|---|---|---|
| | | | | Conf. Int. | |
| | | Std. | Asympt. | Lower | Upper |
| Variables | AUC | Error | Sig | Bound | Bound |
| AngHx + ECG | 0.728 | 0.027 | 0.000 | 0.676 | 0.781 |
| AngHx + ECG + IGFBP-3 | 0.767 | 0.027 | 0.000 | 0.714 | 0.820 |
| AngHx + ECG + | 0.784 | 0.024 | 0.000 | 0.737 | 0.831 |
| HxDyslipid + IGFBP-3 | | | | | |
| AngHx + ECG + | 0.797 | 0.024 | 0.000 | 0.750 | 0.843 |
| HxDyslipid + HDL + | | | | | |
| IGFBP-3 | | | | | |

TABLE 7
Paired Sample Area Difference Under ROC Curves

| | | | | | Asympt. 95% | |
|---|---|---|---|---|---|---|
| | Asympt. | | | Std. | Conf. Int. | |
| | | Sig | AUC | Error | Lower | Upper |
| Test Result Pairs | z | (2-tail) | Diff. | Diff. | Bound | Bound |
| AngHx + ECG | −2.461 | 0.014 | −0.039 | 0.230 | −0.069 | −0.008 |
| versus AngHx + | | | | | | |
| ECG + IGFBP-3 | | | | | | |
| AngHx + ECG | −2.831 | 0.005 | −0.056 | 0.224 | −0.094 | −0.017 |
| versus AngHx + | | | | | | |
| ECG + HxDyslipd + | | | | | | |
| IGFBP-3 | | | | | | |
| AngHx + ECG | −3.583 | 0.000 | −0.068 | 0.223 | −0.106 | −0.031 |
| versus AngHx + | | | | | | |
| ECG + HxDyslipd + | | | | | | |
| HDL + IGFBP-3 | | | | | | |
| AngHx + ECG + | −2.083 | 0.037 | −0.013 | 0.216 | −0.024 | −0.001 |
| IGFBP-3 versus | | | | | | |
| AngHx + ECG + | | | | | | |
| HxDyslipd + | | | | | | |
| HDL + IGFBP-3 | | | | | | |

### Example 4: Improving the Model to Include ΔIGFBP-3 (0-2 hr) Values in “Space” Patient Cohort

Applicants hypothesised that the relative change in IGFBP-3 levels over an initial two hour period from index presentation (i.e. “ΔIGFBP-3” or “Delta-IGFBP-3”) may have clinical utility. This observation is based in part on the assay measuring a specific isoform of IGFBP-3 and/or a specific proteolytic product of IGFBP-3 cleavage which occurs during cardiac ischemia.

The raw ΔIGFBP-3 median values versus adjudicated diagnosis for various conditions are outlined in Table 8 below. These data reveal a positive ΔIGFBP-3 for unstable angina pectoris relative to all other cardiac interrogations.

TABLE 8
ΔIGFBP-3 Median vs Adjudicated Dxs

| | | Number | Std. |
|---|---|---|---|
| IA_Adjudicated_Discharge_Dx | ΔIGFBP-3 | Patients | Deviation |
| Definite UAP | 152.8200 | 59 | 521.20400 |
| Non-Cardiac Chest Pain | −249.6400 | 162 | 414.11320 |
| NSTEMI | −193.3200 | 56 | 393.79801 |
| Other Cardiac | −54.9700 | 18 | 552.06473 |
| Possible UAP | −238.9750 | 82 | 369.44338 |
| STEMI | −280.4600 | 7 | 298.87320 |
| TOTAL | −187.1400 | 384 | 458.15159 |

TABLE 9
Regression Analysis ΔIGFBP-3

| | Variables in the Equation | | | | | |
|---|---|---|---|---|---|---|
| Step 1^{a} | B | S.E. | Wald | df | Sig. | Exp(B) |
| ECG_Change | 1.283 | 0.404 | 10.069 | 1 | 0.002 | 3.606 |
| HxAng | 1.692 | 0.406 | 17.334 | 1 | 0.000 | 5.432 |
| HxDyslip | 0.850 | 0.507 | 2.811 | 1 | 0.094 | 2.339 |
| Delta-IGFBP-3 | 1.029 | 0.196 | 27.633 | 1 | 0.000 | 2.798 |
| HDL_Log10 | −4.687 | 1.525 | 9.443 | 1 | 0.002 | 0.009 |
| Constant | −3.713 | 0.565 | 43.225 | 1 | 0.000 | 0.024 |

Based on ROC analyses, Applicants demonstrate that a positive ΔIGFBP-3 significantly improves the diagnostic power of current UAP predictors, namely history of angina and abnormal ECG. These data are presented in Table 10 below which shows an increase from AUC(HxAng+ECG)=0.735±0.032 to AUC(HxAng+ECG+ΔIGFBP-3)=0.832±0.027. The AUC was enhanced further by the addition of HDL. Refer also to the ROC curve presented in FIG. 4.

TABLE 10
ROC Curve Classification in ED: T = 0-2 h; ΔIGFBP-3 + Current UAP Dx

| | | | | Asympt. 95% | |
|---|---|---|---|---|---|
| | | | | Conf. Int. | |
| | | Std. | Asympt. | Lower | Upper |
| Variables | AUC | Error | Sig | Bound | Bound |
| AngHx + ECG | 0.716 | 0.031 | 0.000 | 0.673 | 0.797 |
| AngHx + ECG + | 0.782 | 0.029 | 0.000 | 0.778 | 0.886 |
| ΔIGFBP-3 | | | | | |
| AngHx + ECG+ | 0.802 | 0.027 | 0.000 | 0.805 | 0.904 |
| HDL + ΔIGFBP-3 | | | | | |

TABLE 11
Paired Sample Area Difference ROC Curves (ΔIGFBP-3)

| | | | | | Asympt. 95% | |
|---|---|---|---|---|---|---|
| | Asympt. | | | Std. | Conf. Int. | |
| | | Sig | AUC | Error | Lower | Upper |
| Test Result Pairs | z | (2-tail) | Diff. | Diff. | Bound | Bound |
| AngHx + ECG | −3.745 | 0.000 | −0.097 | 0.241 | −0.148 | −0.046 |
| versus AngHx + | | | | | | |
| ECG + ΔIGFBP-3 | | | | | | |
| AngHx + ECG | −4.897 | 0.000 | −0.120 | 0.237 | −0.167 | −0.072 |
| versus AngHx + | | | | | | |
| ECG + HDL + | | | | | | |
| ΔIGFBP-3 | | | | | | |
| AngHx + ECG + | −1.955 | 0.051 | −0.022 | 0.227 | −0.045 | 5.677E−5 |
| ΔIGFBP-3 versus | | | | | | |
| AngHx + ECG + | | | | | | |
| HDL + ΔIGFBP-3 | | | | | | |

### Example 5: IGFBP-3 Improves Existing DX of Acute Coronary Syndromes

SPACE study, n=116 ACS cases (57MI, 59UAP) out of 360 total. Significant predictors of acute coronary syndromes at T=0 are hsTnT, HxMI, HxHF and ECG results. Adding IGFBP-3 to risk factor panel significantly increases the ROC from 0.82 to 0.86 (sig.=0.009). NT-proBNP does not achieve this. The results are presented in Table 12 below.

TABLE 12
IGFBP-3 Enhances Dx of Acute Coronary Syndromes

| | | | | Asympt. 95% | |
|---|---|---|---|---|---|
| | | | | Conf. Int. | |
| | | Std. | Asympt. | Lower | Upper |
| Variables | AUC | Error | Sig | Bound | Bound |
| hsTnT + ECG + | 0.821 | 0.024 | 0.000 | 0.774 | 0.868 |
| HxHF + HxMI | | | | | |
| hsTnT + ECG + HxHF + | 0.858 | 0.021 | 0.000 | 0.817 | 0.899 |
| HxMI + IGFBP-3 | | | | | |

These preliminary data have led the Applicants to conclude that IGFBP-3 could provide further improvements in rapid triage of ACS patients and/or to determine whether stress testing might be required for prospective ACS patients.

### Example 6: Utility of ΔIGFBP-3 to Identify Inducible Ischaemia in “Basel VIII” Patient Cohort

Consecutive patients referred to the University Hospital Basel for the evaluation of suspected exercise-induced myocardial ischemia by rest/stress MPI were enrolled. MPI is the preferred imaging modality at this institution for patients with a wide range of pretest probability for exercise-induced myocardial ischemia including patients with and without known cardiovascular disease. Patients were included irrespective of the stress test modality applied (i.e. exercise or pharmacological stress using adenosine or dobutamine). For these analyses, patients were excluded with known clinically relevant structural heart disease other than cardiovascular disease, including heart failure, hypertensive heart disease, moderate or severe valvular dysfunction, atrial fibrillation/flutter, and patients with terminal kidney failure on chronic dialysis, as these conditions were known to result in BNP and hs-cTn release independent from myocardial ischemia. Patients were also excluded if study blood draws or the assessment of clinical judgment were not available.

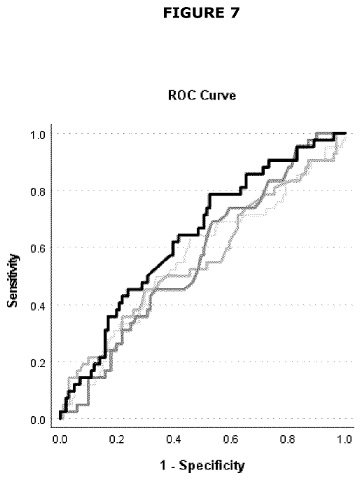

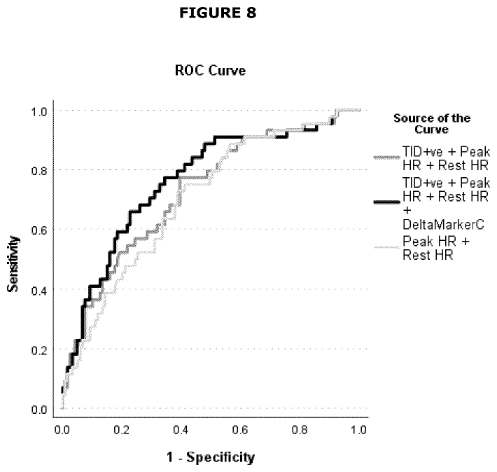

The results of the analyses are presented in Tables 13 and 14 below, read in conjunction with the ROC curves presented in FIGS. 7 and 8.

The data presented in Table 13, read in conjunction with FIG. 7, demonstrate that IGFBP-3 (AUC=0.638±0.050) was a superior biomarker compared to hsTnI (AUC=0.557±0.055), NT-proBNP (AUC=0.563±0.051) and delta-GDF15 (AUC=0.564±0.054) for the identification of inducible ischaemia following stress testing.

TABLE 13
ROC Curve Classification in BASEL VIII:
VARIOUS MARKERS IN ISOLATION

| | | | | Asympt. 95% | |
|---|---|---|---|---|---|
| | | | | Conf. Int. | |
| | | Std. | Asympt. | Lower | Upper |
| Variables | AUC | Error | Sig | Bound | Bound |
| IGFBP-3 | 0.638 | 0.050 | 0.005 | 0.541 | 0.736 |
| hsTnI | 0.557 | 0.055 | 0.298 | 0.449 | 0.665 |
| NT-proBNP | 0.563 | 0.051 | 0.214 | 0.463 | 0.663 |
| ΔGD15 | 0.564 | 0.054 | 0.054 | 0.457 | 0.671 |

However, when ΔIGFBP-3 was considered alongside peak heart rate, resting heart rate and confirmed ischaemia (TID+ve=confirmed transient inducible ischaemia), as measured using spectral or ultrasound imaging, a statistically significant biomarker panel was established for the identification of inducible ischaemia (AUC=0.763±0.043).

TABLE 14
ROC Curve Classification in BASEL VIII:
T = 0-2 h; ΔIGFBP-3 + HR + IMAGING

| | | | | Asympt. 95% | |
|---|---|---|---|---|---|
| | | | | Conf. Int. | |
| | | Std. | Asympt. | Lower | Upper |
| Variables | AUC | Error | Sig | Bound | Bound |
| Peak Heart Rate + | 0.702 | 0.045 | 0.000 | 0.614 | 0.790 |
| Rest Heart Rate | | | | | |
| TID + ve + | 0.725 | 0.045 | 0.000 | 0.637 | 0.812 |
| Peak HR + Rest HR | | | | | |
| TID + ve + Peak HR + | 0.763 | 0.043 | 0.000 | 0.679 | 0.847 |
| Rest HR + ΔIGFBP-3 | | | | | |

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

### REFERENCES

1. Munro A R, Jerram T, Morton T, Hamilton S, 2015. Use of an accelerated diagnostic pathway allows rapid and safe discharge of 70% of chest pain patients from the emergency department. NZ Med J 128:62-71.
2. Ministry of Health, 2012. Mortality and demographic data 2009. Wellington: New Zealand.
3. Thygesen K, Alpert J S, Jaffe A S, Simoons M L, Chaitman B R, White H D; Joint ESC/ACCF/AHA/WHFTask Force for the Universal Definition of Myocardial Infarction, 2012. Third Universal Definition of Myocardial Infarction. Circulation 126:2020-2035.
4. eichlin T, Hochholzer W, Bassetti S, Steuer S, Stelzig C, Hartwiger S, Biedert S, Schaub N, Buerge C, Potocki M, Noveanu M, Breidthardt T, Twerenbold R, Winkler K, Bingisser R, Mueller C, 2009. Early diagnosis of myocardial infarction with sensitive cardiac troponin assays. New England J. Med. 361:858-867.
5. Keller T, Zeller T, Peetz D, Tzikas S, Roth A, Czyz E, Bickel C, Baldus S, Warnholtz A, Fröhlich M, Sinning C R, Lackner K J, Münzel T F, Blankenberg S, 2009. Sensitive troponin I assay in early diagnosis of myocardial infarction. New England J. Med. 361:868-877.
6. Mueller C, 2014. Biomarkers and acute coronary syndromes: an update. Eur. Heart J. 35:552-556.
7. Deckers J W, 2013. Classification of myocardial infarction and unstable angina: a re-assessment.

Int. J. Cardiol. 167:2387-2390.
8. Ranke M B, Traunecker R, Martin D D, Schweizer R, Schwarze C P, Wollmann H A, Binder G, 2005. IGF-I and IGF binding protein-3 levels during initial G H dosage step-up are indicators of G H sensitivity in GH-deficient children and short children born small for gestational age. Horm Res. 64(2):68-76
9. Huang S S, Ling T Y, Tseng W F, Huang Y H, Tang F M, Leal S M, Huang J S, 2003. Cellular growth inhibition by IGFBP-3 and TGF-beta1 requires LRP-1. FASEB J. 17(14):2068-81
10. Ingermann A R, Yang Y F, Han J, Mikami A, Garza A E, Mohanraj L, Fan L, Idowu M, Ware J L, Kim H S, Lee D Y, Oh Y, 2010. Identification of a novel cell death receptor mediating IGFBP-3-induced anti-tumor effects in breast and prostate cancer. J. Biol. Chem. 285(39):30233-46
11. Micutkova L, Hermann M, Offterdinger M, Hess M W, Matscheski A, Pircher H, Mück C, Ebner H L, Laich A, Ferrando-May E, Zwerschke W, Huber L A, Jansen-Dürr P, 2012. Analysis of the cellular uptake and nuclear delivery of insulin-like growth factor binding protein-3 in human osteosarcoma cells. Int. J. Cancer 130(7):1544-57
12. Catapano A L, Graham I, De Backer G, et al. 2016 ESC/EAS guidelines for the management of dyslipidemias. Eur Heart J. 2016; 37:2999-3058

1. A method for treating unstable angina pectoris in a patient who has presented with a history of angina and an abnormal electrocardiogram, the method comprising:
(i) determining a delta insulin-like growth factor binding protein-3 (ΔIGFBP-3) value which comprises:
(a) measuring the concentration of IGFBP-3 from a first sample obtained from the patient;
(b) measuring the concentration of IGFBP-3 from a second sample obtained from the patient at a time point after (a); and
(c) determining the concentration difference in IGFBP-3 between the first and second samples to obtain a ΔIGFBP-3 value
(ii) comparing the ΔIGFBP-3 value from the patient sample against a reference standard of ΔIGFBP-3 from a control population, and
(iii) administering to the patient aspirin, a nitrate, a beta-blocker, a calcium channel blocker, a cholesterol lowering medication, an angiotensin-converting enzyme inhibitor, ranolazine, or any combination thereof.

2. The method according to claim 1, wherein the patient has a decreased heart rate relative to a reference standard.

3. The method according to claim 1, wherein the patient has a decreased levels of high-density lipoprotein relative to a reference standard.

4. The method according to claim 3, wherein the high-density lipoprotein (HDL) is HDL-C.

5. The method according to claim 1, wherein the patient has a diagnosis of ischaemia optionally using spectral imaging or ultrasound imaging.

6. The method according to claim 1, wherein the patient has dyslipidemia or a history of dyslipidemia.

7. The method according to claim 1, wherein the patient has any combination of a decreased heart rate relative to a reference standard, decreased levels of high-density lipoprotein relative to a reference standard, a diagnosis of ischaemia optionally using spectral imaging or ultrasound imaging, and dyslipidemia or a history of dyslipidemia.

8. The method according to claim 1, wherein step (b) is performed between about half an hour to about 10 hours after step (a).

9. The method according claim 8, wherein step (b) is performed about two hours after step (a).

10. The method according claim 1, wherein the patient has, or has a history of, chest pain.

11. The method according to claim 1, wherein the IGFPB-3 is defined by SEQ ID NO: 1 or any fragment thereof.

12. The method according to claim 11, wherein the fragment of IGFBP-3 is a proteolytic cleavage fragment.

13. A method comprising:
(i) measuring a resting heart rate level in a patient suspected of having inducible ischaemia;
(ii) subjecting the patient to a stress test;
(iii) measuring a peak heart rate level in the patient during the stress test;
(iv) determining a delta insulin-like growth factor binding protein-3 (ΔIGFBP-3) value which comprises:
(a) measuring the concentration of IGFBP-3 from a first sample obtained from the patient before the stress test;
(b) measuring the concentration of IGFBP-3 from a second sample obtained from the patient after the stress test;
(c) determining the concentration difference in IGFBP-3 between the first and second samples to obtain a ΔIGFBP-3 value; and
(d) comparing the ΔIGFBP-3 value from the patient sample against a reference standard of ΔIGFBP-3 from a control population.

14. (canceled)

15. The method according to claim 13, wherein the patient receives a prophylactic or therapeutic treatment for ischaemia.

16. A method for detecting a peptide complex from a biological sample, wherein the peptide complex comprises insulin-like growth factor binding protein 3 (IGFBP-3) bound to a monoclonal antibody or antigen-binding fragment thereof which selectively binds to IGFBP-3, and the method comprises detecting IGFBP-3 bound to the monoclonal antibody or antigen-binding fragment and wherein the biological sample is derived from a patient having one or more of a history of angina, a decreased heart rate relative to a reference standard, a decreased level of high-density lipoprotein relative to a reference standard, an abnormal electrocardiogram, a diagnosis of ischaemia, and dyslipidemia or a history of dyslipidemia.

17.-19. (canceled)

20. The method according to claim 1, wherein the levels of IGFBP-3 are measured using an immunoassay.

21. The method according to claim 20, wherein the immunoassay comprises a monoclonal antibody which selectively binds to IGFBP-3 or an antigenic fragment thereof.

22. The method according to claim 13, wherein the levels of IGFBP-3 are measured using an immunoassay.

23. The method according to claim 22, wherein the immunoassay comprises a monoclonal antibody which selectively binds to IGFBP-3 or an antigenic fragment thereof.

24. The method according to claim 1, wherein the sample is selected from plasma, serum, whole blood, arterial blood, venous blood, saliva, bone marrow tissue, heart tissue, vascular tissue, and interstitial fluid.

25. The method according to claim 1, wherein the ΔIGFBP-3 value is at least two, at least three, at least four, at least five, at least six, at least seven, at least eight, at least nine or at least ten times higher than the ΔIGFBP-3 value from the control population.

26. The method according to claim 13, wherein the sample is selected from plasma, serum, whole blood, arterial blood, venous blood, saliva, bone marrow tissue, heart tissue, vascular tissue, and interstitial fluid.

27. The method according to claim 13, wherein the stress test is induced using an exercise stress test or a pharmacological stress test.

28. The method according to claim 27, wherein the exercise induced stress test is performed using a stationary exercise bicycle, a treadmill, a stepping machine and/or a rowing machine.

29. The method according to claim 27, wherein the pharmacological stress test is induced by administering adenosine or dopamine to the patient.

30. The method according to claim 15, wherein the treatment is aspirin, a nitrate, a beta-blocker, a calcium channel blocker, a cholesterol lowering medication, an angiotensin-converting enzyme inhibitor, ranolazine, or any combination thereof.


SEQUENCE LISTING (2 sequences)

SEQ ID NO: 1 - protein, 264 aa, unidentified
GASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 2 - protein, 27 aa, unidentified
MQRARPTLWAAALTLLVLLRGPPVARA